SEQUENCE LISTING (26 sequences)

SEQ ID NO: 1 - protein, 15 aa, Homo sapiens
KVEKIGEGTYGVVYK

SEQ ID NO: 2 - protein, 15 aa, Homo sapiens
FNDVYLVTHLMGADL

SEQ ID NO: 3 - protein, 15 aa, Homo sapiens
FSEVYLVTTLMGADL

SEQ ID NO: 4 - protein, 15 aa, Homo sapiens
FTDFYLVMPFMGTDL

SEQ ID NO: 5 - protein, 15 aa, Homo sapiens
FYDFYLVMPFMQTDL

SEQ ID NO: 6 - protein, 15 aa, Homo sapiens
FQDVYIVMELMDANL

SEQ ID NO: 7 - protein, 15 aa, Homo sapiens
MKDVYIVQDLMETDL

SEQ ID NO: 8 - protein, 15 aa, Homo sapiens
GKQYWLITAFHAKGN

SEQ ID NO: 9 - protein, 15 aa, Homo sapiens
TSTVQLITQLMPFGC

SEQ ID NO: 10 - protein, 15 aa, Homo sapiens
GGPIYIITEYCPYGD

SEQ ID NO: 11 - protein, 15 aa, Homo sapiens
QEPIYIITEYNENGB

SEQ ID NO: 12 - protein, 15 aa, Homo sapiens
EEPIYIVTEYMSKGS

SEQ ID NO: 13 - protein, 15 aa, Homo sapiens
EPPFYIITEFMTYGN

SEQ ID NO: 14 - protein, 15 aa, Homo sapiens
WTQLWLVSDYHEHGS

SEQ ID NO: 15 - protein, 15 aa, Dictyostelium discoideum
FEDYYIVSELMDTDL

SEQ ID NO: 16 - protein, 15 aa, Caenorhabditis elegans
IEDVYFVSMLMGADL

SEQ ID NO: 17 - protein, 15 aa, Caenorhabditis elegans
LNNVYFVSVLMGSDL

SEQ ID NO: 18 - protein, 15 aa, mammalian
WTQLWLVSDYHEHGS

SEQ ID NO: 19 - protein, 15 aa, mammalian
GKQYWLITAFHAKGN

SEQ ID NO: 20 - protein, 15 aa, mammalian
STQLWLITHYHEMGS

SEQ ID NO: 21 - protein, 15 aa, mammalian
WTQLWLVSDYHEHGS

SEQ ID NO: 22 - protein, 15 aa, mammalian
DVDLWLITAFHEKGS

SEQ ID NO: 23 - protein, 15 aa, mammalian
EVELWLITAFHDKGS

SEQ ID NO: 24 - protein, 15 aa, mammalian
WTQLYLITDYHENGS

SEQ ID NO: 25 - protein, 15 aa, mammalian
WTQLYLITDYHENGS

SEQ ID NO: 26 - protein, 15 aa, mammalian
RMEYLLVMEYYPNGS

(19) United States
(12) Patent Application Publication
(10) Pub. No.: US 2005/0234094 A1
(43) Pub. Date: Oct. 20, 2005

(54) Inhibition of protein kinases with piridinylimidazoles

(75) Inventors: Philip Cohen, Dundee (GB); Michel Goedert, Cambridge (GB); Patrick Alexander Eyers, Dundee (GB)

Correspondence Address: GLAXOSMITHKLINE;Corporate Intellectual Property - UW2220, P.O. Box 1539, King of Prussia, PA, 19406-0939, US

(73) Assignee: SmithKline Beecham p.l.c.

(21) Appl. No.: 11/030,487

(22) Filed: Jan. 6, 2005

Related U.S. Application Data
(63) Continuation of application No. 09/700,253, filed on Apr. 22, 2001.

(30) Foreign Application Priority Data
May 9, 1998 (GB) 9809869.2

(51) Int. Cl. A61K 31/4745; C12Q 1/48

(52) U.S. Cl. 514/303; 435/15

(57) ABSTRACT

a method of inhibiting a protein kinase that has a threonine or less bulky residue at the position equivalent to Thr 106 in SAPK2a/p38, and not mammalian SAPK2a/p38 or SAPK2b/p38β2, is provided, wherein the protein kinase is exposed to a pyridinyl imidazole inhibitor. The protein kinase may be a TGFβ type-I or type-II receptor.

The present invention relates to the inhibition of protein kinases by pyridinylimidazoles.

Compounds belonging to the class of pyridinylimidazoles have been found to be inhibitors of cytokine expression and are known as CSAIDs (cytokine-suppressive anti-inflammatory drugs). They have been found to inhibit SAPK2a/p38 and SAPK2b/p38β2 MAP kinases (proteins involved in intracellular signal transduction) but not to inhibit many other protein kinases, including MAP kinases that are very closely related. The basis of this specificity has been investigated by crystallographic studies and/or mutagenesis by Tong et al (1997) Nature Structural Biology 4(4), 311-316, Wilson et al (1997) Chemistry & Biology 4, 423-431 and by kinetic studies by Young et al (1997) J Biol Chem 272, 12116.

Tong et al (1997) and Wilson et al (1997) both discuss the role of the residue at the position equivalent to that of Thr 106 in SAPK2a/p38 in the binding of pyridinylimidazole inhibitors. Tong et al concluded that this residue is not critical on the basis that p38γ (SAPK3) is sensitive to these inhibitors, whereas Wilson et al recognised that p38γ is insensitive to these inhibitors, as shown by Cuenda et al (1997) EMBO J. 16, 295-305.

Wilson et al constructed mutants of SAPK2a/p38 which were insensitive to pyridinylimidazole inhibitors, and concluded that the size of the residue in the position equivalent to that of Thr 106 in SAPK2a/p38 is important in determining inhibition of MAP kinases, though other residues are also discussed. Neither Tong et al or Wilson et al discuss selecting protein kinases for which pyridinylimidazole inhibitors may be suitable “lead” inhibitors.

It was not appreciated that pyridinyl imidazoles could be useful lead compounds for the development of compounds that are selective inhibitors of other particular kinases. The present work surprisingly shows that kinases selected solely on the basis of lack of a bulky hydrophobic residue at the critical position may be inhibited by pyridinylimidazole inhibitors. Such kinases include a type-II TGFβ receptor, Src family members (particularly Lck) and a type-I TGFβ receptor, and may further include type-I and type-II activin, TGFβ and bone morphogenetic protein (BMP) receptors. The present work further shows that the potency of inhibition of a type-I TGFβ receptor is much higher than that of the type-II receptor. These findings may allow identification and development of pyridinylimidazole inhibitors or derivatives thereof that may be useful as selective modulators of the signalling of TGFβ and other members of the families of TGFβ ligands (such as activins and bone morphogenetic proteins; BMPs) via the family of TGFβ/activin/BMP receptors. The new knowledge concerning protein kinases inhibited by known pyridinylimidazole inhibitors, such as SB 203580, may allow use of the known inhibitors for different purposes, for example exploiting the inhibition of the type-I TGFβ receptor.

The TGFβ family, signalling pathways and likely functions have been extensively researched and reviewed. TGFβ appears to be involved in the modulation of many biological processes, and may be implicated in pathogenic conditions including tumour growth, inflammation, wound healing, scarring, fibrosis, kidney damage, for example in diabetes, and atherosclerosis. Proteins related to TGFβ include activins, inhibins and bone morphogenetic proteins (BMPs). In some situations, enhancement of TGFβ signalling may be beneficial, whilst in others, inhibition may be useful. A lack of specific small-molecule agonists or antagonists of TGFβ signalling has impeded investigations, particularly in vivo.

The views expressed in a selection of reviews are summarised below.

Attisano L; Wrana J L (1996) “Signal transduction by members of the transforming growth factor-β superfamily.” Cytokine Growth Factor Rev 7(4), 327-39 describes the receptor complexes of transmembrane serine/threonine kinases through which transforming growth factor-β (TGF β) superfamily members exert their diverse biological effects. Both components of the receptor complex, known as receptor I (type-I) and receptor II (type-II) are essential for signal transduction. The composition of these complexes can vary significantly due to the promiscuous nature of the ligands and the receptors, and this diversity of interactions can yield a variety of biological responses. Several receptor interacting proteins and potential mediators of signal transduction have now been identified, including Mothers against dpp-related (MADR or SMAD) proteins.

ten Dijke P; Franzen P; Yamashita H; Ichijo H; Heldin C H; Miyazono K (1994) “Serine/threonine kinase receptors.” Prog Growth Factor Res 5(1), 55-72 discusses transmembrane receptors that contain intracellular serine/threonine kinase domains. Ligands for this class of receptors include members of the transforming growth factor-β (TGF-β) superfamily, e.g. TGF-βs and activins. TGF-βs exert their effects on target cells via formation of heteromeric serine/threonine kinase complexes (TGF-β type I and type II receptors). Other components, i.e. TGF-β type III receptor and endoglin, appear to have more indirect roles, e.g. to present ligands to the signalling receptors. Given the structural similarity between members of the TGF-β superfamily, other ligands in this family may act through structurally and functionally similar serine/threonine kinase receptors.

Hartsough M T; Mulder K M (1997) “Transforming growth factor-β signalling in epithelial cells” Pharmacol Ther 75 (1), 21-41 discusses the resistance of some tumours to growth suppression by TGFβ. It also discusses signalling pathways (not yet fully elucidated), including the SMAD signalling components that control TGF-β-mediated gene transcription.

Heldin C H; Miyazono K; ten Dijke P (1997) “TGF-β signalling from cell membrane to nucleus through SMAD proteins.” Nature 390, 465-71 further discusses the involvement of the SMAD family of signal transducer proteins. Pathway-restricted SMADs are held to be phosphorylated by specific cell-surface receptors that have serine/threonine kinase activity, then to oligomerise with the common mediator Smad4 and translocate to the nucleus where they direct transcription to effect the cell's response to TGF-β. Inhibitory SMADs have been identified that block the activation of these pathway-restricted SMADs.

Noble N A; Border W A (1997) “Angiotensin II in renal fibrosis: should TGF-β rather than blood pressure be the therapeutic target?” Semin Nephrol 17(5), 455-66 discusses the role of TGFβ in promoting tissue fibrosis and the induction of TGFβ by angiotensin II.

Koli K; Keski-Oja J (1996) “Transforming growth factor-β system and its regulation by members of the steroid-thyroid hormone superfamily.” Adv Cancer Res 70, 63-94, discusses TGF-βs and their receptors and their action as key regulators of many aspects of cell growth, differentiation, and function, particularly malignancy.

Grande J P (1997) “Role of transforming growth factor-β in tissue injury and repair.” Proc Soc Exp Biol Med 214(1), 2740 discusses the role of TGFβ in normal cell growth, development, and tissue remodelling following injury. Disruption of the TGFβ1 gene in utero produces a wasting syndrome characterised by systemic inflammation, suggesting that this growth factor plays an important role in limiting the inflammatory response. TGFβ is a dominant mediator of the pathologic extracellular matrix accumulation that characterises progression of tissue injury to end-stage organ failure. Recent studies directed towards characterisation of the TGFβ genes, dissection of the mechanisms by which TGFβs are produced and activated, and identification of TGFβ signalling pathways have established the important roles that these family members play in cell and tissue homeostasis. TGFβ structure-function relationships and their relevance to models of tissue injury/wound repair are also discussed.

Lawrence D A (1996) “Transforming growth factor-β: a general review.” Eur Cytokine Netw 7(3), 363-74 reviews the roles of TGF-β1, β2 and β3 in mammals. The author comments that they play critical roles in growth regulation and development. All three of these growth factors are secreted by most cell types, generally in a latent form, requiring activation before they can exert biological activity. This activation of latent TGF-β, which may involve plasmin, thrombospondin and possibly acidic microenvironments, appears to be a crucial regulatory step in controlling their effects. The TGF-βs possess three major activities: they inhibit proliferation of most cells, but can stimulate the growth of some mesenchymal cells; they exert immunosuppressive effects; and they enhance the formation of extracellular matrix. Two types of membrane receptors (type I and type II) possessing a serine/threonine kinase activity within their cytoplasmic domains are involved in signal transduction. Inhibition of growth by the TGF-βs stems from a blockage of the cell cycle in late G1 phase. Among the molecular participants concerned in G1-arrest are the Retinoblastoma (Rb) protein and members of the Cyclin/Cyclin-dependent kinase/Cyclin dependent kinase inhibitor families. In the intact organism the TGF-βs are involved in wound repair processes and in starting inflammatory reactions and then in their resolution. The latter effects of the TGF-βs derive in part from their chemotactic attraction of inflammatory cells and of fibroblasts. From gene knockout and from overexpression studies it has been shown that precise regulation of each isoform is essential for survival, at least in the long term. Several clinical applications for certain isoforms have already shown their efficacy and they have been implicated in numerous other pathological situations.

Pignatelli M; Gilligan C J (1996) “Transforming growth factor-β in G1 neoplasia, wound healing and immune response.” Baillieres Clin Gastroenterol 10(1), 65-81 discusses the influence that cell-cell and cell-matrix interactions, the differentiating status of the cell together with the functional activity of other soluble growth factors have on responses to TGF-βs, particularly in relation to homeostasis of the GI mucosa and their role in gastrointestinal carcinogenesis.

Cox D A (1995) “Transforming growth factor-β 3.” Cell Biol Int 19(5), 357-71 discusses the molecular and cellular biology of TGF-β 3 and those physiological actions which may lead to clinical applications, particularly in the indication areas of wound healing and chemoprotection.

Wahl S M (19920 “Transforming growth factor β (TGF-β) in inflammation: a cause and a cure.” J Clin Immunol 12(2), 61-74 discuses the mechanisms controlling whether the pro- or antiinflammatory effects of this peptide prevail.

Ruscetti F W; Palladino M A (1991) “Transforming growth factor-β and the immune system.” Prog Growth Factor Res 3(2), 159-75 discusses the increased levels of TGF-β found in several disease states associated with immunosuppression such as different forms of malignancy, chronic degenerative diseases, and AIDS, implicating the involvement of TGF-β in the pathogenesis of some diseases.

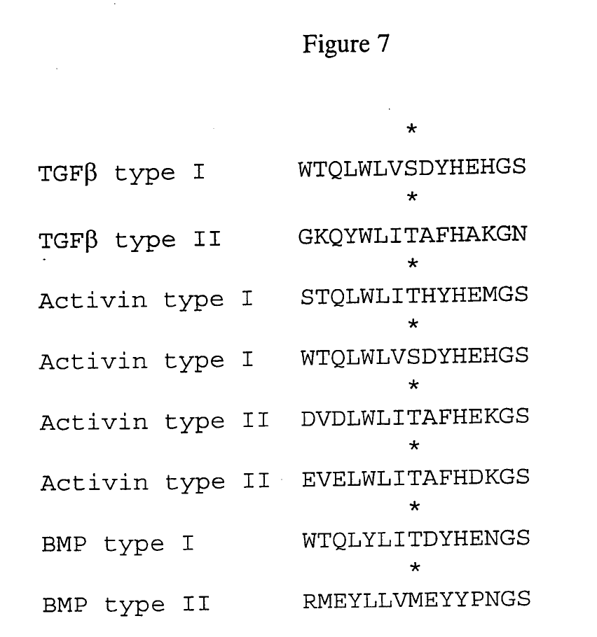

An alignment of partial sequences (including the position equivalent to Thr is 106 in SAPK2a/p38) of TGFβ, activin and BMP type-I and type-II receptors is shown in FIG. 7. The cloning of the type-I TGFβ receptor is described in Franzén et al (1993) Cell 74, 681-692. The cloning of the closely related type-I activin receptor is described in Cárcamo et al (1994) Mol Cell Biol 14(6), 3810-3821, which also reviews other members of the type-I and type-II receptor families. The cellular responses to TGFβ and activin mediated respectively by these related receptors appear to be similar, and may include growth inhibition and stimulation of extracellular matrix protein expression.

The type-I TGFβ and activin receptors both have a serine residue at the position equivalent to Thr 106 of SAPK2a/p38. With the exception of a type-II BMP receptor, type-I and type-II receptors may have a serine or threonine (another residue less bulky than methionine) at the position equivalent to Thr 106 in SAPK2a/p38.

Law & Lydon “The anticancer potential of tyrosine kinase inhibitors” Chapter 12 of “Emerging drugs: the prospect for improved medicines. Annual Executive Briefing 1996”, Ashley Publications Ltd, reviews the implication of tyrosine kinases, including the epidermal growth factor receptor (EGFR) family, platelet derived growth factor receptor (PDGFR) family, Src family (including Lck and Fyn), Abl and the Philadelphia chromosome fusion gene Bcr-Abl, in various cancers. Tyrosine kinases may also be involved in other pathological states. Lck, for instance, is a T-cell specific kinase; inhibition of Lck may lead to immunosuppression.

Pyridinylimidazole inhibitors have been suggested to be useful in the treatment of various diseases or conditions on the basis of in vitro effects on cytokine production, for example as discussed in WO 95/02591. The diseases or conditions were selected as those in which IL-1, tumour necrosis factor (TNF) or IL-8 were thought to play a role. No evidence of efficacy is presented in any of the diseases or conditions. Compounds of Formula I (which encompasses pyridinyl imidazoles plus many other compounds) are said, for example on page 49 (lines 1 to 10), to be useful in methods of treating diseases/conditions have been selected on the basis of being conditions that would benefit from inhibition of inflammation by inhibiting proinflammatory cytokines, particularly IL-1, IL-6, IL-8 and TNF. It would be clear to a reader of WO 95/02591 that the compounds were only expected to be of benefit in forms of the diseases listed in which excessive inflammation was thought to be involved.

Badger et al (1996) J Pharmacol Exp Ther 279(3), 1453-1461 describes the use of a pyridinylimidazole inhibitor in animal models of arthritis, bone resorbtion and endotoxin shock, conditions selected on the basis of the involvement of TNF-α and possibly IL-1, both proinflammatory cytokines. This paper refers to other studies of the use of pyridinylimidazole inhibitors in models of inflammatory disease.

TGFβ is known to be an inhibitor of inflammation (as reviewed, for example, in Lawrence (1996) and Grande (1997), both cited above) for example from studies in which massive inflammatory lesions are seen in mice in which a TGFβ gene is inactivated.

The previously supposed mode of action of pyridinylimidazole inhibitors provides no motivation to use these inhibitors in diseases or conditions in which it would be desirable to reduce an anti-inflammatory effect (as may be caused by TGFβ), or in diseases or conditions shown to involve TGFβ (for example in causing or exacerbating the disease or condition). It also provides no motivation to use pyridinylimidazole inhibitors in diseases or conditions which had previously been suggested to be suitable for treatment with these inhibitors but which more recent research has now shown not to be caused by excessive inflammation.

The present invention relates to inhibition of protein kinases which contain a threonine or less bulky residue, preferably a Serine residue, at the position equivalent to Thr 106 in SAPK2a/p38 by pyridinylimidazole inhibitors.

Thus, a first aspect of the invention is a method of inhibiting a protein kinase that has a threonine or less bulky residue at the position equivalent to Thr 106 in SAPK2a/p38 wherein the protein kinase is exposed to a pyridinylimidazole inhibitor or related inhibitor, provided that the protein kinase is not mammalian SAPK2a/p38 or SAPK2b/p38β2.

A second aspect of the invention is the use of a pyridinylimidazole inhibitor or related inhibitor in a method of inhibiting a protein kinase that has a threonine or less bulky residue at the position equivalent to Thr 106 in SAPK2a/p38 wherein the protein kinase is exposed to a pyridinylimidazole inhibitor or related inhibitor, provided that the protein kinase is not mammalian SAPK2a/p38 or SAPK2b/p38β2.

A third aspect of the invention is a screening method for identifying a drug-like compound or lead compound for the development of a drug-like compound in which (1) a pyridinylimidazole or related compound is exposed to a protein kinase that has a threonine or less bulky amino acid at the position equivalent to Thr 106 in SAPK2a/p38 and is not mammalian SAPK2a/p38 or SAPK2b/p38β2 and (2) the binding of the compound to the protein kinase is measured or the change in the activity of the protein kinase is measured.

It is preferred that the pyridinylimidazole is a pyridinylimidazole inhibitor, as defined below.

The term “drug-like compound” is well known to those skilled in the art, and may include the meaning of a compound that has characteristics that may make it suitable for use in medicine, for example as the active ingredient in a medicament. Thus, for example, a drug-like compound may be a molecule that may be synthesised by the techniques of organic chemistry, less preferably by techniques of molecular biology or biochemistry, and is preferably a small molecule, which may be of less than 5000 daltons molecular weight. A drug-like compound may additionally exhibit features of selective interaction with a particular protein or proteins and be bioavailable and/or able to penetrate cellular membranes, but it will be appreciated that these features are not essential.

The term “lead compound” is similarly well known to those skilled in the art, and may include the meaning that the compound, whilst not itself suitable for use as a drug (for example because it is only weakly potent against its intended target, non-selective in its action, unstable, difficult to synthesise or has poor bioavailability) may provide a starting-point for the design of other compounds that may have more desirable characteristics.

The use or methods may be performed in vitro, either in intact cells or tissues, with broken cell or tissue preparations or at least partially purified components. Alternatively, they may be performed in vivo. The cells tissues or organisms in/on which the use or methods are performed may be transgenic. In particular they may be transgenic for the protein kinase under consideration or for a further protein kinase.

A further aspect of the invention is a compound identifiable or identified by the said screening method. It will be appreciated that such a compound may be an inhibitor of the protein kinase used in the screen and that the intention of the screen is to identify compounds that act as inhibitors of the protein kinase, even if the screen makes use of a binding assay rather than an enzymic activity assay. It will be appreciated that the inhibitory action of a compound found to bind the protein kinase may be confirmed by performing an assay of enzymic activity in the presence of the compound.

A further aspect of the invention is a method of determining that a protein kinase is sensitive to a pyridinylimidazole inhibitor, comprising comparing the amino acid sequence or three dimensional structure of the protein kinase with that of SAPK2a/p38 and determining that the protein kinase has a threonine or less bulky residue at the position equivalent to Thr 106 in SAPK2a/p38.

The comparison of amino acid sequences or three dimension structure (for example from crystallography or computer modelling based on a known structure) may be carried out using methods well known to the skilled man, as detailed below.

The following preferences apply to all appropriate aspects of the invention.

The amino acid at the position equivalent to Thr 106 in SAPK2a/p38 may be, for example, threonine, serine, alanine or glycine. Preferably it is serine.

The protein kinase is preferably a naturally occuring kinase or variant thereof. By this meaning is included that the amino acid at the position equivalent to Thr 106 in SAPK2a/p38 is naturally a threonine or less bulky residue, and is not a threonine or less bulky residue as a consequence of in vitro mutation of the polynucleotide encoding the kinase.

It will be appreciated that the polynucleotide encoding the protein kinase may be mutated in order to encode a variant of the protein kinase, for example by insertion, deletion, substitution, truncation or fusion, as known to those skilled in the art. It is preferred that the protein kinase is not mutated in a way that may materially affect its biological behaviour, for example its enzymatic activity.

The protein kinase may be a type-II TGFβ receptor (a serine/threonine protein kinase); Lin et al (1992) Cell 68, 775), type-I (Attisano et al (1993) Cell 75, 671-680) or type-II activin receptor or type-I BMP receptor (see Cárcamo et al (1994) Mol Cell Biol 14(6), 3810-3821), Src family (protein tyrosine kinase) member (particularly Lck; Tevilyan et al (1986) Biochem Biophys Acta 888, 286), Abl, or a receptor protein tyrosine kinase, such as epidermal growth factor (EGF) receptor or platelet derived growth factor (PDGF) receptor. These proteins have a threonine residue at the position equivalent to Thr 106 in SAPK2a/p38.

The protein kinase may be type-I TGFβ receptor (Franzén et al (1993) Cell 75, 681-692) or type-I activin receptor (Cárcamo et al (1994). These protein kinases have a serine residue at the position equivalent to Thr 106 in SAPK2/p38.

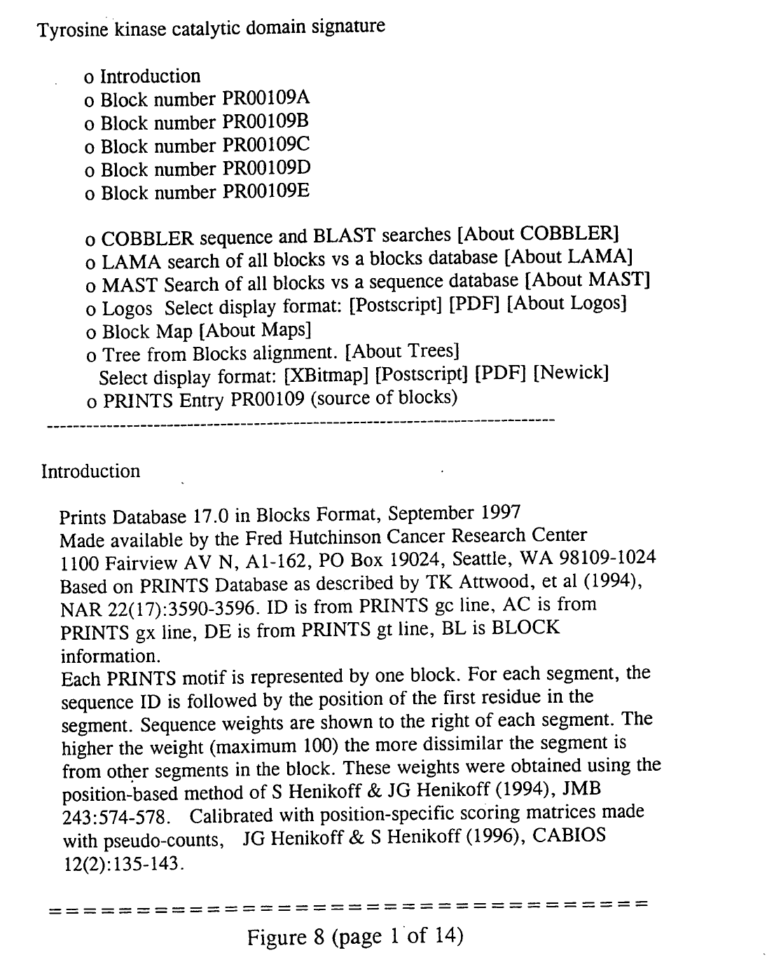

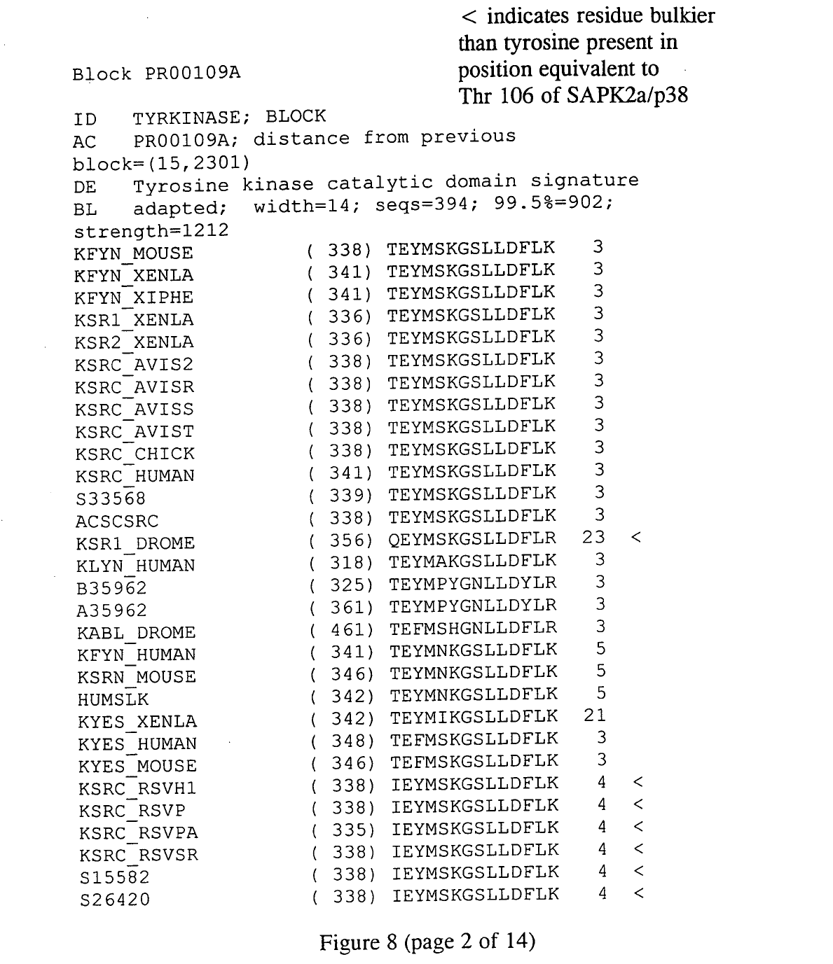

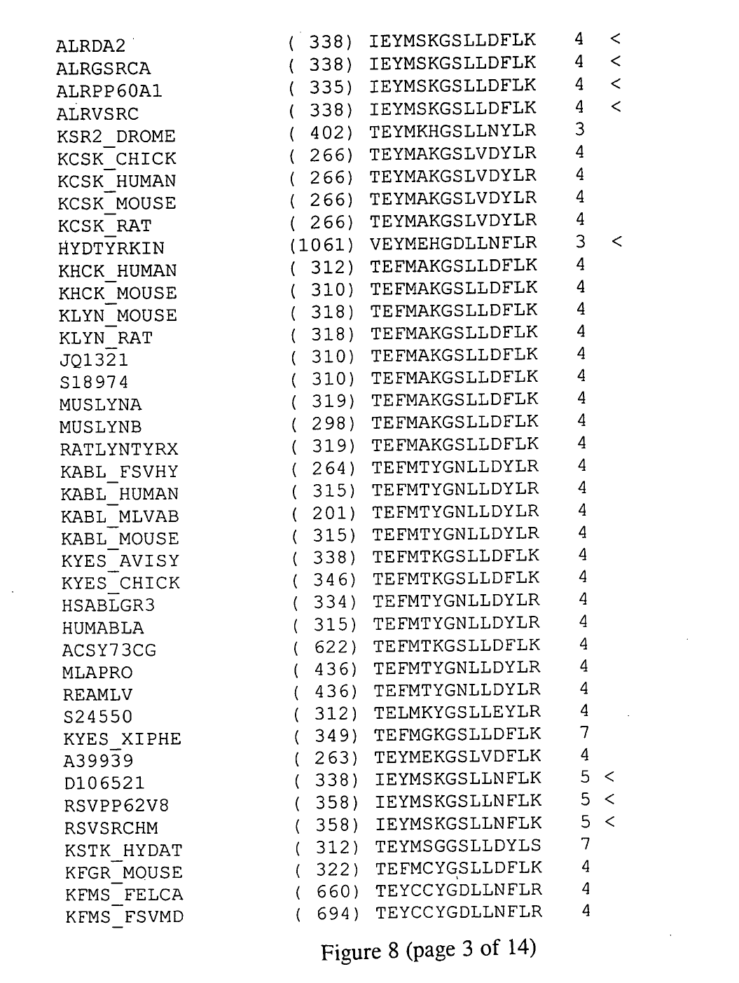

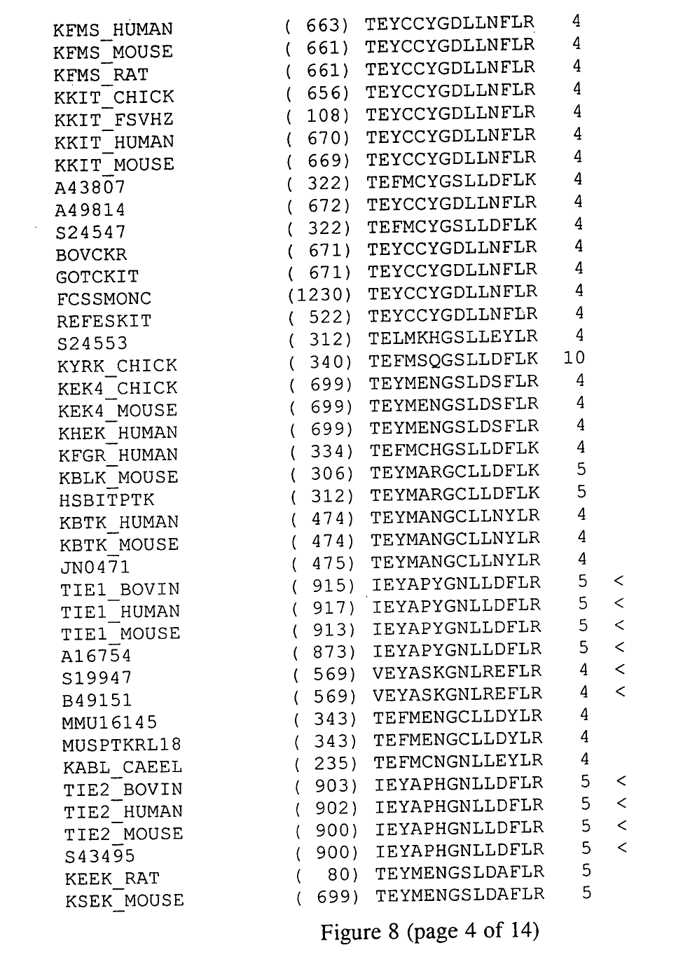

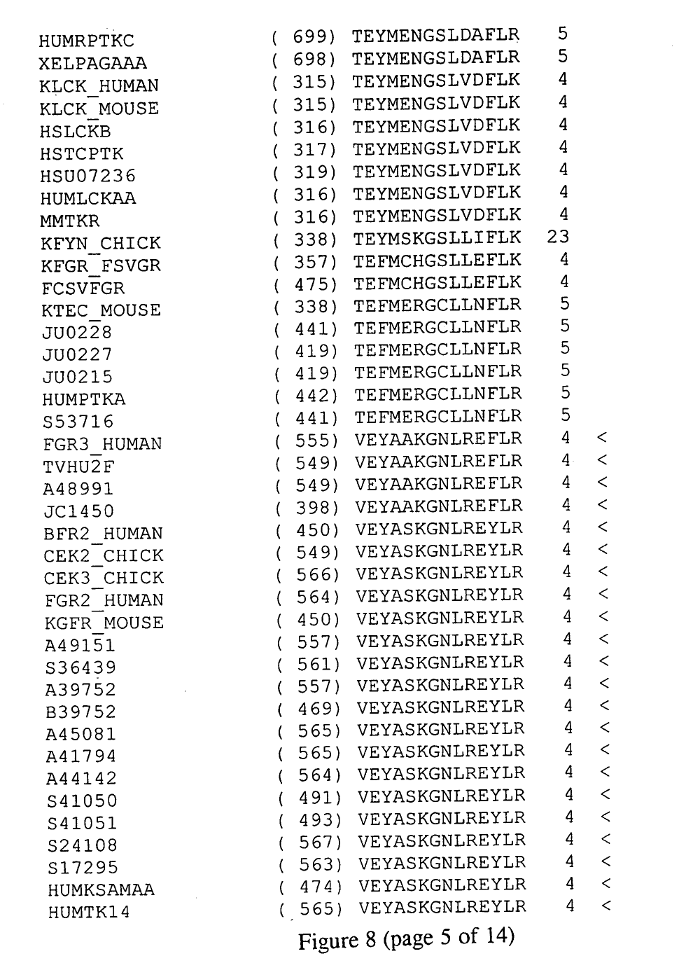

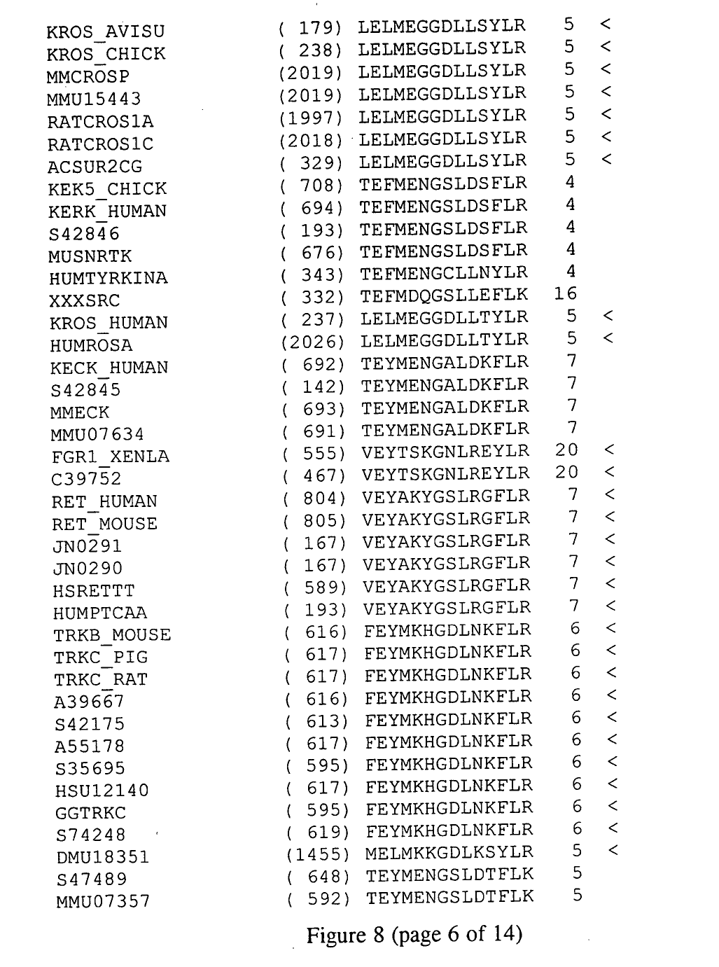

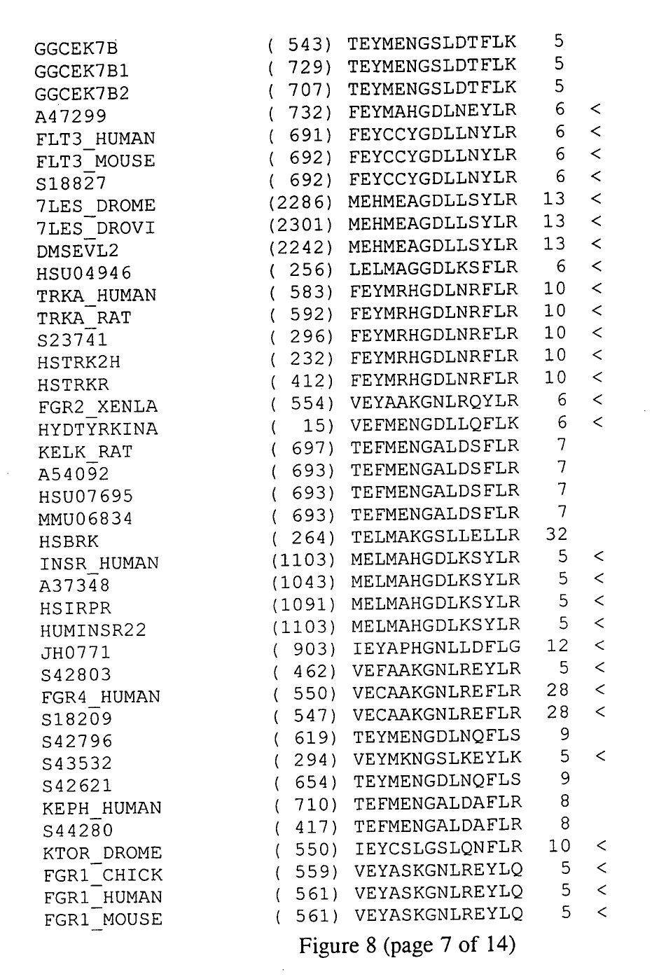

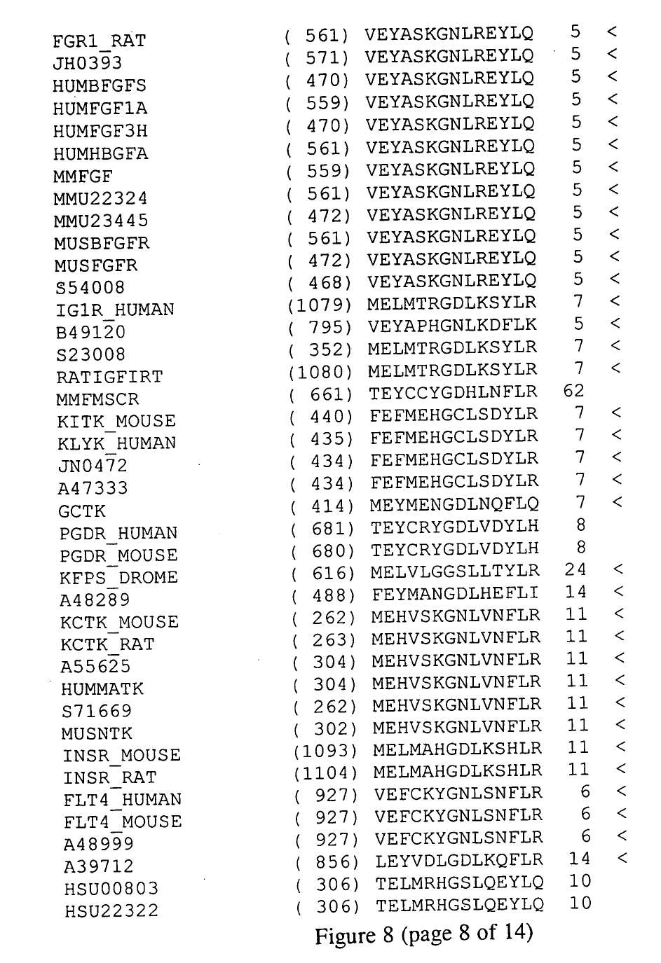

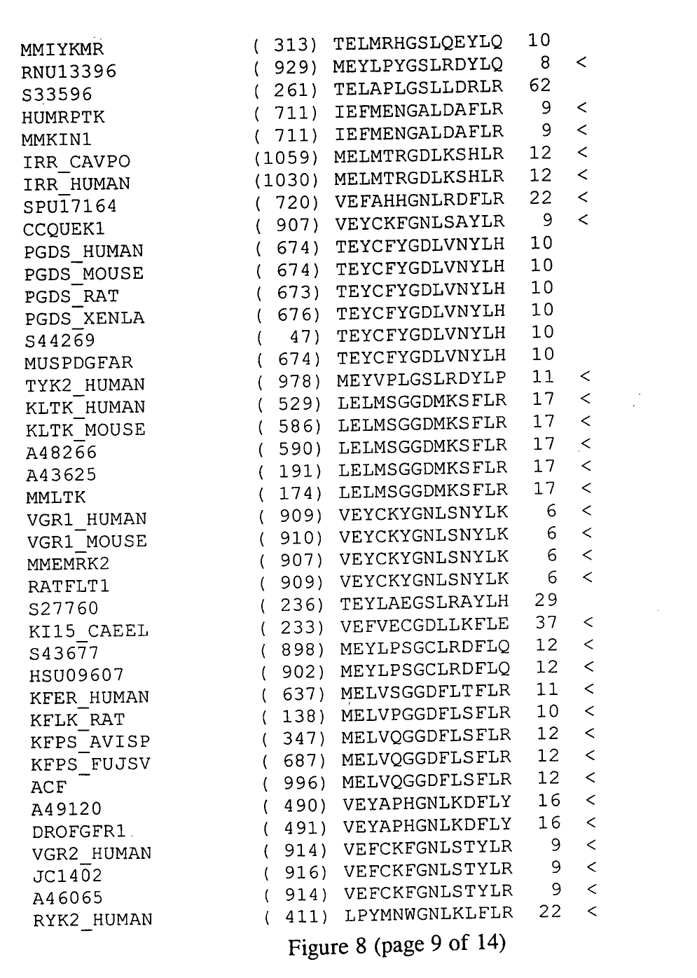

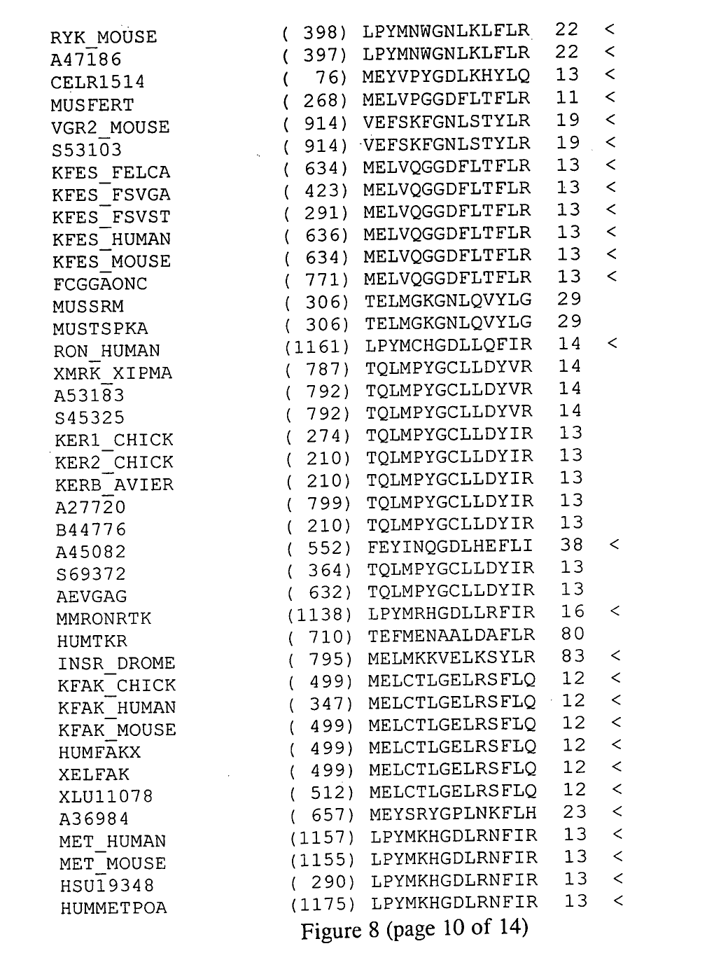

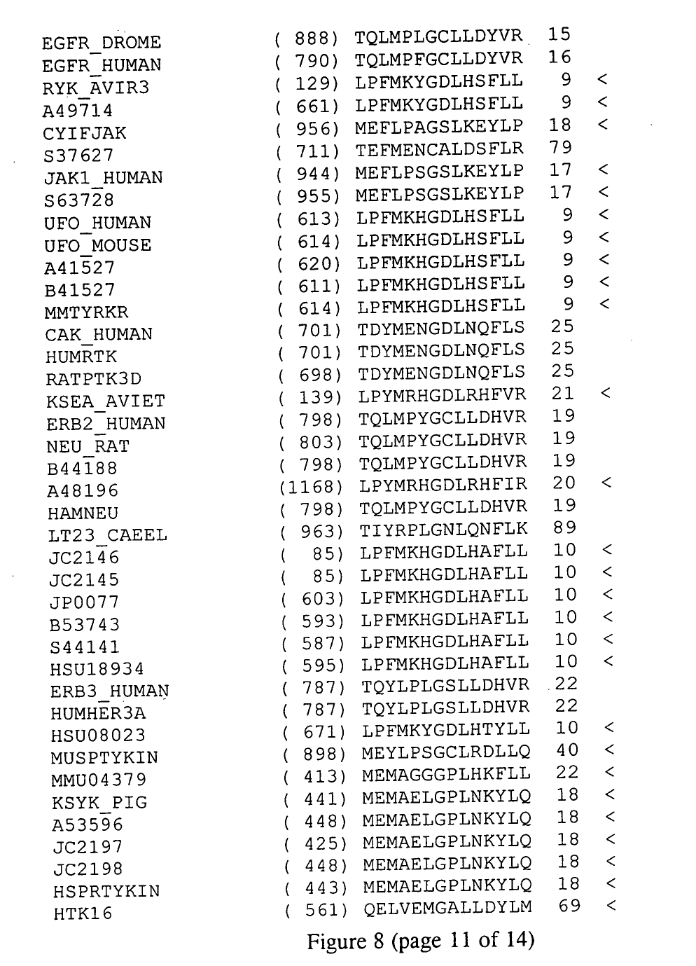

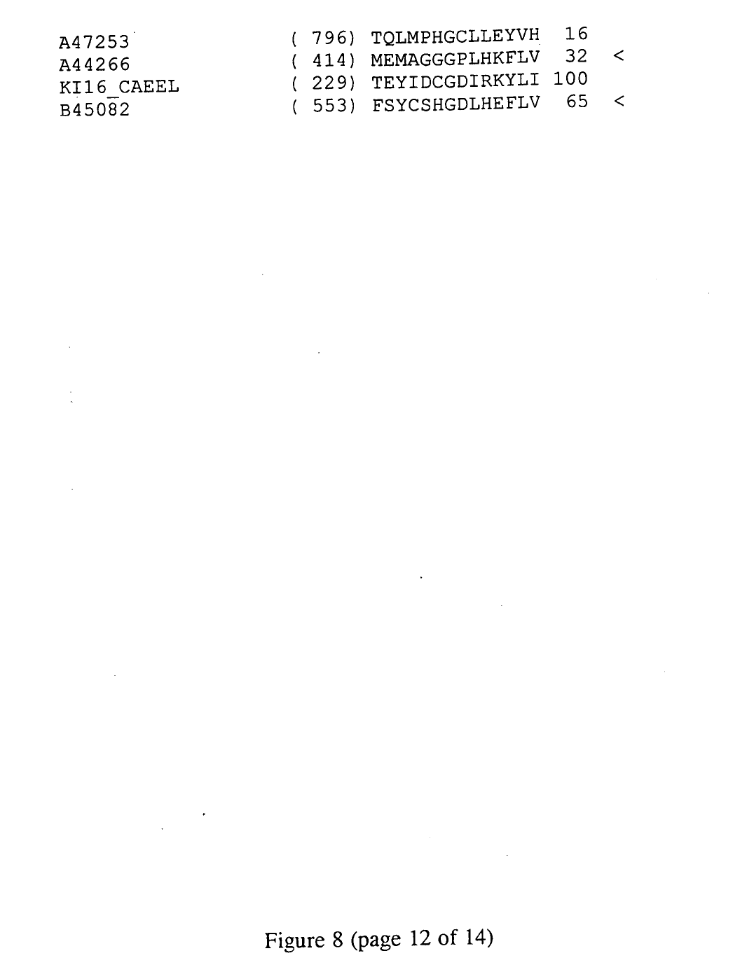

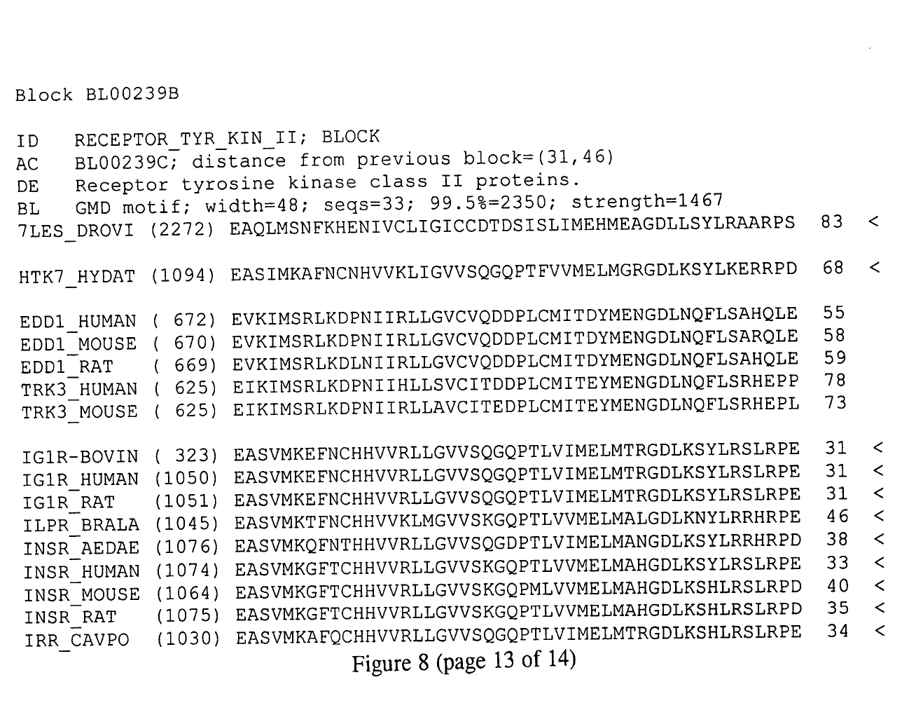

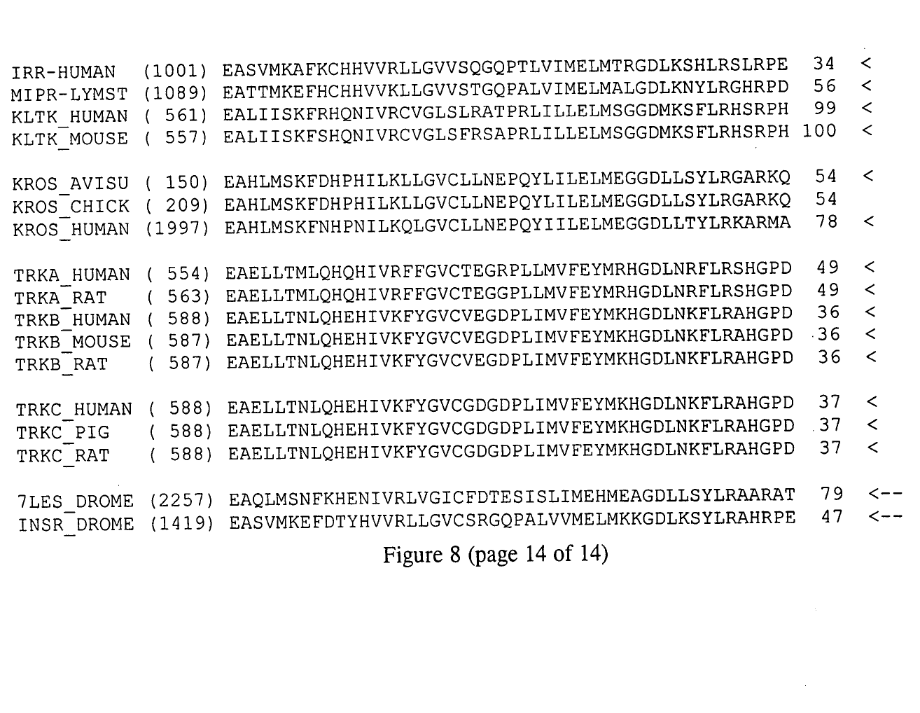

Further protein kinases with a threonine or less bulky residue at the position equivalent to Thr 106 in SAPK2a/p38 are shown in FIG. 8, which shows partial sequences including the residue equivalent to Thr 106 in SAPK2a/p38 of tyrosine kinase sequences held in the Prints Database 17.0. The information shown in FIG. 8 is sufficient to allow identification of the records relating to the partial sequences shown.

A TGF-β type I receptor appears to be slightly more sensitive to a pyridinyl imidazole inhibitor. As discussed in detail in Example 1, the IC₅₀ for inhibition of the type-I receptor by SB 203580 is 20 μM. The IC₅₀ for inhibition of the Type-II receptor is 40 μM. It is particularly preferred that the protein kinase is a type-I TGFβ receptor.

The present finding, as discussed in Example 1, that sensitivity to SB 203580 can be gained by a single amino acid substitution means that it may be possible to exploit the same drug to identify the physiological roles of all MAP kinase family members and other protein kinases. For example, it may be investigated whether all the reported effects of SB 203580 in mammalian cells are abolished by transfection with a drug-insensitive form of SAPK2a/p38 or by the use of transgenic mice expressing an SB 203580-insensitive form of this protein kinase. The further replacement of wild-type SAPK1/JNK, SAPK3 or SAPK4 by drug-sensitive forms of these enzymes in transgenic mice expressing a drug-resistant form of SAPK2a/p38 may also be useful in addressing the physiological roles of these other MAP kinase family members.

A further aspect of the invention is the use of a transgenic animal or a transfected cell in a method of determining a physiological role of a protein kinase, wherein the cell or animal comprises a protein kinase that has been mutated at the position equivalent to Thr 106 in SAPK2a/p38.

The protein kinase for which the role is to be determined may be a MAP kinase family member or other protein kinase. The protein kinase for which the role is to be determined may or may not be the protein kinase that is mutated in the transgenic animal or transfected cell. The animal is preferably a rodent, still more preferably a mouse. The cell may be from a mammal, preferably human.

It will be appreciated that “investigating the physiological, role or roles of a protein kinase” does not comprise merely expressing the mutant protein kinase in a cell for the purposes of subsequent purification and/or determination of the sensitivity of the mutant protein kinase to inhibition by a compound, for example a pyridinylimidazole inhibitor.

A still further aspect of the invention is the use of a transgenic animal, for example a mouse or other rodent, or transfected cells, comprising a protein kinase that has been mutated at the position equivalent to Thr 106 in SAPK2a/p38, in a screening method for identifying a substrate of said protein kinase.

A further aspect of the invention comprises a transgenic animal, for example a mouse or other rodent, comprising a protein kinase that has been mutated at the position equivalent to Thr 106 in SAPK2a/p38.

Methods of constructing such cells or transgenic animals are well known to those skilled in the art. It will be appreciated that the protein kinase mutated at the position equivalent to Thr 106 in SAPK2a/p38 may also be mutated in other ways, for example by insertion, deletion, truncation or fusion, as known to those skilled in the art. It is preferred that the protein kinase is not mutated in a way that may materially affect its biological behaviour, for example its enzymatic activity.

It will be appreciated that the protein kinase may be mutated to replace a residue bulkier than threonine with threonine or a less bulky residue, or to replace threonine or a less bulky residue with a residue bulkier than threonine, for example methionine.

By “equivalent of Thr 106 in SAPK2a/p38” is meant the amino acid residue that occupies a position in the native three dimensional structure of a protein kinase corresponding to the position occupied by Thr 106 in the native three dimensional structure of human SAPK2a/p38, for example as described in Tong et al (1997) or Wilson et al (1997) and the crystal structures referred to in those papers. It will be appreciated that Thr 106 of SAPK2a/p38 lies within the ATP binding pocket of SAPK2a/p38 but in a part of the binding pocket that is not utilised by ATP.

Protein kinases show a conserved catalytic core, as reviewed in Johnson et al (1996) Cell, 85, 149-158 and Taylor & Radzio-Andzelm (1994) Structure 2, 345-355. This core folds into a small N-terminal lobe largely comprising anti-parallel β-sheet, and a large C-terminal lobe which is mostly α-helical. A deep cleft at the interface between these lobes is the site of ATP binding, with the phosphate groups near the opening of the cleft.

Protein kinases also show conserved sequences within this catalytic core, and the residue equivalent to Thr 106 in SAPK2a/p38 may be identified by alignment of the sequence of the kinase with that of known kinases in such a way as to maximise the match between the sequences. The alignment may be carried out by visual inspection and/or by the use of suitable computer programs, for example the GAP program of the University of Wisconsin Genetic Computing Group, which will also allow the percent identity of the polypeptides to be calculated. The Align program (Pearson (1994) in: Methods in Molecular Biology, Computer Analysis of Sequence Data, Part II (Griffin, A M and Griffin, H G eds) pp 365-389, Humana Press, Clifton).

The sequence for SAPK2a/p38 is given, for example, in Goedert et al. (1997) EMBO J. 16, 3563-3571 and Lee et al (1994) Nature 372, 739-746.

It will be appreciated that the residue equivalent to Thr 106 of SAPK2a/p38 is not itself well conserved between protein kinases. A bulky residue is present at this position in many kinases (see, for example, Hanks et al (1988) Science 241, 42-52), for example glutamine in ERK2 and methionine in p38γ, but a smaller residue is present in some kinases, for example threonine in SAPK2b/p38β. Thus identification of the residue equivalent to Thr 106 will depend on the alignment of surrounding conserved residues.

The residue at the position equivalent to Leu 104 in SAPK2a/p38 may also be important in determining whether a protein kinase may be inhibited by a pyridinylimidazole inhibitor (or related inhibitor), as described in Example 1. It is preferred that the protein kinase does not have an isoleucine residue at this position. It is further preferred that the protein kinase has a leucine residue at this position.

If the protein kinase has a leucine at the position equivalent to Leu 104 in SAPK2a/p38, it may be inhibited by a pyridinylimidazole inhibitor (or related inhibitor) even if the residue at the position equivalent to Thr 106 in SAPK2a/p38 is methionine (for example, JNK2β1 and JNK2β2 (Whitmarsh et al (1997) Mol Cell Biol 17, 2360). Thus, the type-II BMP receptor whose sequence is shown in FIG. 7 may also be inhibited by a pyridinylimidazole inhibitor.

It will be appreciated that the various aspects of the invention that apply in relation to a protein kinase with a threonine or less bulky residue at the position equivalent to Thr 106 in SAPK2a/p38 may also apply (with appropriate modification) in relation to a protein kinase with a methionine or less bulky residue at the position equivalent to Thr 106 and a leucine (or not an isoleucine) at the position equivalent to Leu 104 in SAPK2a/p38, that is not mammalian SAPK2a/p38, SAPK2b/p38β2, JNK2 βl or JNK2β2.

The following assays may be useful in a screening method as set out above.

Interaction of a compound with a protein kinase may be measured by measuring inhibition of the enzymatic activity of the protein kinase or by measuring the association/dissociation of the compound from the protein kinase. For example, the fluorescence emission spectrum of a protein may change upon the binding of a compound. Such an assay, where the emission of SAPK2a/p38 in response to excitation at 280 nm is partially shifted from 335 nm to 375 nm upon binding of VK-19911, is described in Wilson et al (1997).

Examples of enzymatic assays are described in Example 1 below. For example, myelin basic protein may be used as a substrate for SAP kinases a peptide substrate such as KVEKIGEGTYGVVYK may be used for Lck and histone H₂B may be used as a substrate for type-Il TGFβ receptor and other type-II receptors. Autophosphorylation may be measured for the type-I TGFβ receptor and other type-I receptors (see also Cárcamo et al (1994)). MADR proteins may be phosphorylated by type-I receptors, as reviewed in Attisano & Wrana (1996), cited above, and may therefore be suitable as substrates for type-I receptors. MADR2 and MADR3, for example, may be phosphorylated by a TGFβ type-I receptor.

It will be appreciated that the phosphorylation of the chosen substrate may be measured using techniques known to those skilled in the art. For example, detection may be by using labelled (eg radiolabelled; ³²P or ³³P) phosphate in free solution or attached to the substrate, and comparing the amount associated with (or dissociated from) the substrate before and after the assay.

For tyrosine kinases, phosphorylation of the substrate tyrosine may be measured by detecting a change in fluorescence when the tyrosine is phosphorylated or dephosphorylated, as is known to those skilled in the art.

Some of the assay components may be localised on a surface, such as a blotting membrane, or an assay plate for ELISA etc, although the assay may be carried out in solution.

It will be appreciated that the assay may be carried out in vitro, for example with purified or partly purified components, or may be performed using whole cells, or may be carried out in vivo.

It is preferred that the assay is capable of being performed in a “high throughput” format. This may require substantial automation of the assay and minimisation of the quantity of a particular reagent or reagents required for each individual assay. A scintillation proximity assay (SPA) based system, as known to those skilled in the art, may be beneficial.

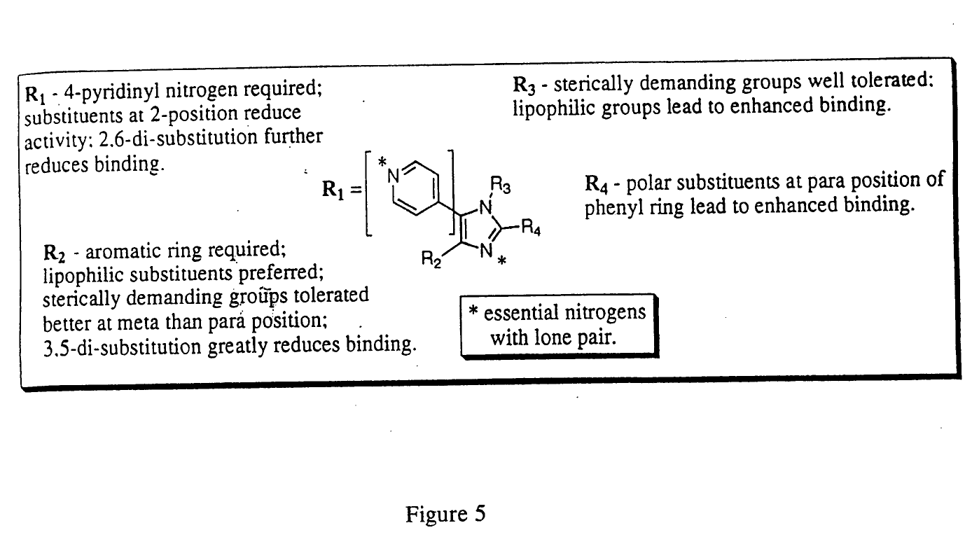

The term “pyridinylimidazole inhibitor” is well known to those skilled in the art, and encompasses compounds comprising a pyridyl ring and an imidazole ring with substituents as shown in FIG. 5 and as described below, which bind to and/or inhibit SAPK2a/p38 (or less preferably, are known to inhibit IL-1 production from monocytes) as set out in Gallagher et al (1997) Bioorg Med Chem 5(1), 49-64. Compounds of this type are known inhibitors of particular protein kinases and as cytokine-suppressive anti-inflammatory drugs (CSAIDs). Use of these compounds in investigating signalling pathways is reviewed in Cohen (1997) Trends Cell Biol 7, 354-361.

It will be appreciated that the numerical measure of binding affinity or inhibition IC₅₀ for a particular compound/protein combination will depend upon the exact assay system used. A pyridinylimidazole is considered to be a pyridinylimidazole inhibitor if it has a binding IC₅₀ or kinase IC₅₀ for SAPK2a/p38 (CSBP) of less than 100 μM, preferably less than 10 μM, still more preferably less than 1 or 0.1 or 0.01 μM as set out in Gallagher et al or in the assay as set out in Example 1, preferably as determined in the assay as set out in Example 1.

It will be appreciated that it is expected that such a pyridinylimidazole inhibitor may also inhibit a protein kinase with a threonine or less bulky amino acid residue in the position equivalent to Thr 106 of SAPK2a/p38, particularly a type-I TGFβ receptor. It will further be appreciated that the pyridinylimidazole inhibitor may alternatively (but less preferably) be defined by the ability to inhibit a protein kinase with a threonine or less bulky amino acid residue in the position equivalent to Thr 106 of SAPK2a/p38, particularly a type-I TGFβ or activin receptor, which protein kinase is not mammalian SAPK2a/p38 or SAPK2b/p38β2.

With reference to FIG. 5, a 4-pyridinyl nitrogen is required in group R₁: substituents at the 2-position reduce activity and 2,6 di-substitution further reduces binding. An aromatic ring is required in position R₂: lipophilic substituents are preferred; sterically demanding groups are tolerated better at the meta than the para position, and 3,5 di-substitution greatly reduces binding. Sterically demanding groups are well tolerated at position R₃ and lipophilic groups lead to enhanced binding. At position R₄, polar substituents at the para position of a phenyl ring lead to enhanced binding.

By “pyridinylimidazole compound” is meant a compound comprising a pyridinylimidazole structure, but which may not fulfil the requirements for being a pyridinylimidazole inhibitor, as set out above. For example, it may not bind to or inhibit SAPK2a/p38 at sufficiently low concentration, or said binding or inhibition may not be detectable.

By “related compound” is meant a compound, at least part of which may adopt a conformation substantially similar to those parts of a pyridinylimidazole inhibitor that interact with a protein kinase, for example SAPK2a/p38. This may be determined by molecular modelling, using techniques known to those skilled in the art. Such a compound may be able to bind to and inhibit a protein kinase, preferably a protein kinase with a threonine or less bulky amino acid residue in the position equivalent to Thr 106 of SAPK2a/p38, in a manner substantially similar to a pyridinylimidazole inhibitor.

By a “related inhibitor” is meant a related compound that is able to bind to or inhibit such a protein kinase, preferably not mammalian SAPK2a/p38 or SAPK2b/p38β2, still more preferably a type-I TGFβ receptor. It is preferred that the binding IC₅₀ or kinase IC₅₀ for the interaction of the compound and protein kinase is less than 10 μM, preferably less than 1 μM, still more preferably less than 0.1 or 0.01 μM in an assay as set out in Example 1 or similar to such an assay if a different protein kinase is used.

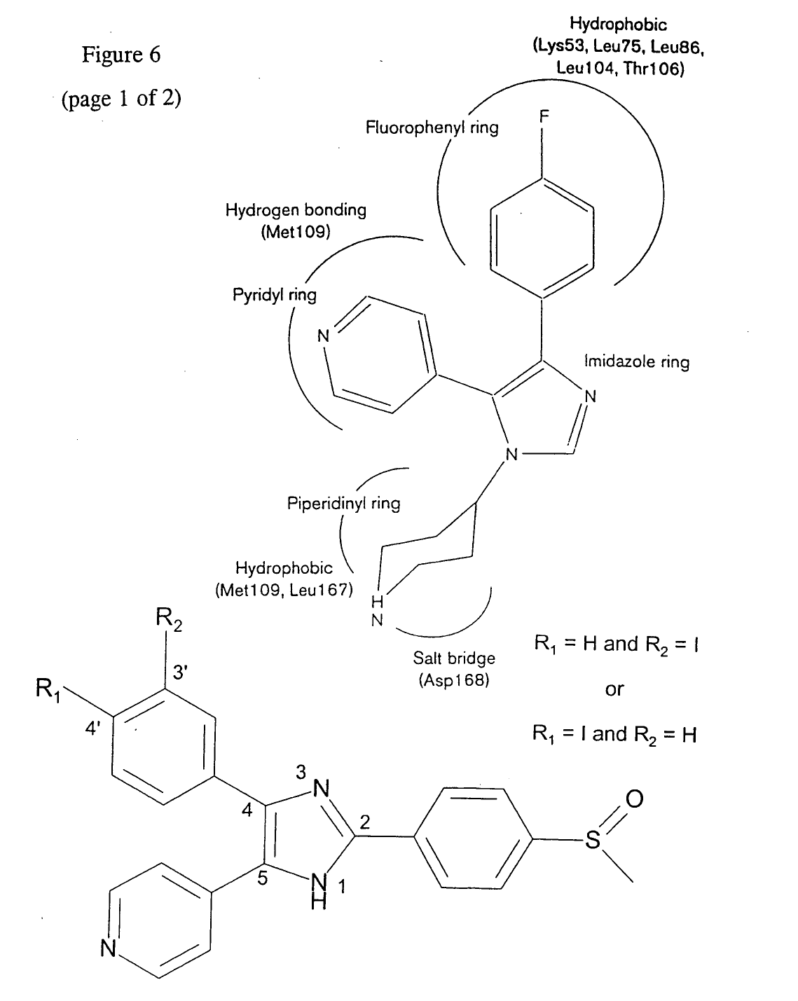

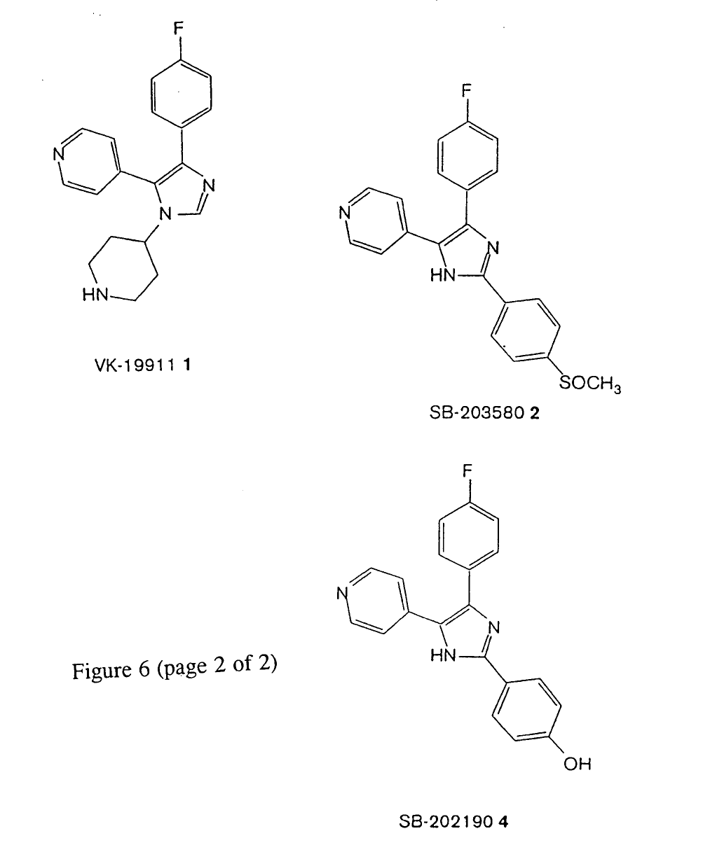

The 4-phenyl ring of the pyridinylimidazole inhibitor may be important in determining the specificity of pyridinylimidazole inhibitors for protein kinases which have a threonine or less bulky residue in the position equivalent to Thr 106 of SAPK2a/p38. The ring may have a fluorine substituent, as in SB 203580, SB 202190, VK-19911 (FIG. 6) or a iodine substituent as described in Tong et al (1997). As set out in Wilson et al (1997), the ortho and meta positions on one face of the ring may be sterically blocked by the backbone carbonyl of Ala 51 and the sidechain of Val 38 respectively of SAPK2a/p38, while the para position, as well as the ortho and meta positions of the opposing face of the ring appear to allow small substituents. Thus monosubsitution at the meta or para position may increase the potency of these compounds.

It will thus be appreciated that it is preferred that the pyridinylimidazole or pyridinylimidazole inhibitor comprises a 4-phenyl ring.

Synthesis of a pyridinylimidazole inhibitor (VK-19911; 4-(4-fluoro-phenyl)-1-(4-piperidinyl)-5-(4-pyridyl)-imidazole) is described, for example in Wilson et al (1997). The following references may also be relevant:

Gallagher et al (1995) “2,4,5-triarylimidazole inhibitors of IL-1 biosynthesis” Bioorg Med Chem Lett 5, 1171-1176; Boehm et al (1996) “1-substituted 4-aryl-Spyridinylimidazoles: a new class of cytokine suppressive drugs with low 5-lipoxygenase and cyclooxygenase inhibitory potency” J Med Chem 39, 3929-3937; Van Leusen et al (1977) “Base-induced cycloaddition of sulfonylmethyl isocyanides to C,N double bonds. Synthesis of 1,5-disubstituted and 1,4,5-trisubstituted imidazoles from aldimines and imidoyl chlorides” J Org Chem 42, 1153-1159; WO 95/02591 (SmithKline Beecham); Mach et al (1993) “¹⁸F-labelled benzamides for studying the dopamine D2 receptor with positron emission tomography” J Med Chem 36, 3707-3719; Lee et al (1993) “Bicyclic imidazoles as a novel class of cytokine biosynthesis inhibitors” Ann N Y Acad Sci 696, 149-170; Lee et al (1994) Nature 372, 739-746.

Structure-function relationships were investigated in Gallagher et al (1995) Bioorg Med Chem Lett 5, 1171-1176 and Boehm et al (1996) J Med Chem 39, 3929-3937.

It is preferred that the pyridinylimidazole inhibitor has the nitrogen atom in the pyridyl ring in the para-position. If the nitrogen atom is moved to the ortho- or meta-position, the potency of the inhibitor may decrease.

Examples of pyridinlyimidazole inhibitors include SB 202190 (a 2,4,5-triarylimidazole), SB 203580 (another 3,4,5-triarylimidazole) and derivatives such as the 3′-iodinated compound as described in Tong et al (1997) (FIG. 6). A derivative in which the p-methylsulphinylphenyl group is removed may also act as an inhibitor. Substituents may be made at the N1 atom of the imidazole ring and substitutions made at the 2-position of the imidazole ring may be moved to the N1 atom without significant loss of potency.

It is preferred that the pyridyl ring is a 4-pyridyl ring. 3-pyridyl or 2-pyridyl derivatives may be unable to form a hydrogen bond with the backbone carbonyl of the residue equivalent to Met 109 of SAPK2a/p38, as described in Wilson et al (1997) and may therefore be less active.

SK&F 105809 is an example of a pyridinylimidazole that does not inhibit SAPK2a/p38 and also does not inhibit the type-II TGFβ receptor (see Example I). This has a sulfoxide moiety at the 4 position of the 4-phenyl ring. The sulfide metabolite, SK&F 105561, is active.

It will be appreciated that the structure-activity relationship observed with kinases against which members of the pyridinylimidazole class of inhibitors have so far been tested may not be the same for other protein kinases. It may be possible to provide and characterise compounds belonging to or based on the pyridinylimidazole class of inhibitors that are able to inhibit certain protein kinases, but not inhibit other protein kinases.

It will be appreciated that a pyridinylimidazole or related compound may be used which may comprise chemical groups that may interact with other parts of the protein kinase molecule. Such groups may enhance the binding of the compound to a particular protein kinase and/or reduce the binding of the compound to a different protein kinase.

SB 203580 may be obtained from Calbiochem, Nottingham, UK. The pharmacological profile of SB 203580 is described in Badger et al (1996) J Pharmacol Exp Ther 279(3), 1453-1461.

Thus, the work presented here shows that it may be possible to predict whether a protein kinase will be sensitive to inhibition by a member of the class of pyridinyl imidazoles, by inspection of the amino acid at the position equivalent to Thr 106 of SAPK2a/p38. It may therefore be possible to identify “lead” inhibitors for a number of protein kinases, with reduced or no requirement for high throughput screening during lead compound identification. These “lead” compounds may then be developed further, for example by molecular modelling/and or experiments to determine the structure activity relationship for inhibitors of a particular protein kinase, in order to develop more efficacious compounds, for example by improving potency, selectivity/specificity and pharmacokinetic properties.

It will be appreciated that a method of identifying specific inhibitors may comprise testing a compound for ability to inhibit 1) a protein kinase with a threonine or less bulky residue at the position equivalent to Thr 106 in SAPK2a/p38, and 2) other protein kinases, which may or may not have a threonine or less bulky residue at the position equivalent to Thr 106 in SAPK2a/p38, or 3) other proteins with a biological activity, in order to investigate the selectivity of the compound for inhibition of the protein kinase specified in 1) above.

The TGFβ type I and type-II receptors may be involved, for example, in scarring, tissue regeneration and kidney response to diabetes and therefore inhibition of either receptor may be useful in medicine. Inhibition of the type-I receptor is preferred. Activin type-I and type-II receptors may be mediate activins' roles in regulating endocrine cells from the reproductive system, promoters of erythroid differentiation and in inducing axial mesoderm and anterior structures in vertebrates. Inhibins may have effects antagonistic to those of activins. BMP receptors may be involved in similar processes to TGFβ and activins, and particularly in bone growth and maintenance. TGFβs may be expressed in a wider range of tissues than other members of the superfamily, which may have more specialised roles.

Tyrosine kinases may be implicated in several disease states, including various cancers, as reviewed in Law & Lydon (1996), cited above. Pyridinylimidazole inhibitors and related inhibitors may therefore be useful in treating cancers associated with the subset of tyrosine kinases defined above, that may be inhibited by such inhibitors.

For example, members of the EGF receptor family may be involved in cancers of epithelial origin, for example breast, lung, ovary or colon. Overexpression of these receptors may be associated with a poor disease outcome. The erbB2 receptor may also have a threonine at the position equivalent to Thr 106 in SAPK2a/p38. The erbB2 receptor is a constitutively active mutant EGF receptor and may be implicated in similar cancers to the EGF receptor. Inhibition of both receptors (for example by pyridinylimidazole or related inhibitors), particularly in combination with hormonal agents for the treatment of hormone-dependent breast and prostate malignancies may be effective, possibly due to synergistic effects of the agents.

Members of the PDGF receptor family may be involved in cancers of mesenchymal (sarcomas) or glial origin, solid tumours including refractory prostate and ovarian cancer, melanoma and Kaposi's sarcoma. The related stem cell factor receptor may be involved in small cell lung cancer.

A fusion protein, Bcr-Abl, encoded by the Philadelphia chromosome (formed by translocation between chromosomes 9 and 22) appears to be involved in chronic myelogenous leukaemia (CML). It is a constitutively active protein tyrosine kinase related to the Abl tyrosine kinase discussed above. An inhibitor of Bcr-Abl may be useful in removing leukaemic cells ex vivo from bone marrow removed from a patient, prior to reintroducing the bone marrow back into the patient.

Src kinase family members have been implicated in several tumour types, including breast cancer (Src; correlating with poor prognosis), colon cancer and T-cell lymphomas (Src, Fyn and Lck).

An inhibitor of a tyrosine kinase involved in tumourigenicity may have a tumouristatic or tumouricidal effect when used in isolation. It is preferred that it has a tumouricidal effect. An inhibitor may be used with another agent, for example a cytotoxic or hormonal agent, as known to those skilled in the art, in order to achieve improved efficacy. An inhibitor able to inhibit more than one tyrosine kinase implicated in cancer may be particularly beneficial.

TGFβ may also be involved in carcinogenesis (see, for example Lawrence (1996), cited above) and therefore pyridinylimidazole and related inhibitors that inhibit TGFβ and related receptors may be useful in the treatment of cancer.

It will be appreciated that in the discussion which follows of diseases and conditions in which pyridinylimidazole inhibitors and related inhibitors may be useful, forms of conditions or diseases understood to be caused by proinflammatory cytokines, in particular IL-1, TNF, IL-6 and IL-8, for which pyridinylimidazoles have previously been suggested to be useful, are excluded. It is preferred that the forms of the conditions or diseases in which pyridinylimidazole inhibitors and related inhibitors may be useful are forms which TGFβ or members of the TGFβ family of proteins may be implicated or involved in causing or exacerbating. It is preferred that TGFβ or activin is involved.

A still further aspect of the invention is the use of a pyridinylimidazole inhibitor or related inhibitor or a compound identifiable by the screening method described above in the manufacture of a medicament for the treatment of a patient in need of inhibition of a protein kinase that has a threonine or less bulky residue at the position equivalent to Thr 106 of SAPK2a/p38, wherein the protein kinase is not SAPK2a/p38 or SAPK2b/p38β2.

It is preferred that the protein kinase has a serine at the position equivalent to Thr 106 of SAPK2a/p38. It is particularly preferred that the protein kinase is a TGFβ or activin receptor, still more preferably a type-I TGFβ or activin receptor.

The protein kinase may be a protein tyrosine kinase, particularly EGF receptor, PDGF receptor, Abl or a Src family kinase. The patient may be a patient with cancer, in particular a cancer with which such a protein tyrosine kinase has been linked. Examples of such cancers include cancers of epithelial origin, for example breast, lung, ovary, colon or prostate, cancers of mesenchymal (sarcomas) or glial origin, solid tumours including refractory prostate and ovarian cancer, melanoma and Kaposi's sarcoma, small cell lung cancer, chronic myelogenous leukaemia (CML), or T-cell lymphomas.

A further aspect of the invention is the use of a pyridinylimidazole inhibitor or related inhibitor or compound identifiable by the screening method described above in the manufacture of a medicament for the treatment of a patient in need of reducing extracellular matrix deposition, encouraging tissue repair and/or regeneration, tissue remodelling or healing of a wound, injury or surgery, or reducing scar tissue formation arising from injury to the brain.

Extracellular matrix deposition is a term well known to those skilled in the art, and is described for example in Grande (1997) and Lawrence (1996), cited above. Extracellular matrix components include collagens, fibronectin, tenascin, glycosaminoglycans and proteoglycans. Deposition of such components may lead to rapid wound healing but may also lead to scarring, particularly in the brain. TGFβ may inhibit degradation of the extracellular matrix (for example by inhibiting production of proteases and stimulating the production of specific protease inhibitors.

It will be appreciated that the medicament may be applied before surgery. It will be appreciated that the injury may be mechanical injury. It is preferred that it is not reperfusion injury.

A still further aspect of the invention is the use a pyridinylimidazole inhibitor or related inhibitor or compound identifiable by the screening method described above in the manufacture of a medicament for the treatment of a patient with or at risk of end-stage organ failure, pathologic extracellular matrix accumulation, a fibrotic condition, disease states associated with immunosuppression (such as different forms of malignancy, chronic degenerative diseases, and AIDS), diabetic nephropathy, tumour growth, kidney damage (for example obstructive neuropathy, IgA nephropathy or non-inflammatory renal disease) or renal fibrosis.

It will be appreciated that forms of chronic degenerative diseases in which excessive IL-1, IL-8 or TNF production is implicated are not disease states associated with immunosuppression, and are therefore excluded from the definition. Thus forms of multiple sclerosis or muscle degeneration in which excessive IL-1, IL-8 or TNF production is implicated are excluded. Similarly, forms of AIDS in which excessive IL-1, IL-8 or TNF production is implicated are excluded.

The patient may alternatively have, or be at risk of, a form of a disorder of bone growth or homeostasis (such as osteoporosis), arthritis or atherosclerosis in which IL-1, IL-6, IL-8 or TNF or a proinflammatory cytokine have not been implicated, but in which TGFβ or a related protein (for example an activin, inhibin or BMP) has been implicated, in causing or exacerbating the condition.

The tumour or malignancy may of a type linked with a particular protein tyrosine kinase, as set out above.

A further aspect of the invention is the use of a pyridinylimidazole inhibitor or related inhibitor or compound identifiable by the screening method described above in the manufacture of a medicament for the treatment of a patient with a cancer of epithelial origin, for example breast, lung, ovary, colon or prostate, a cancer of mesenchymal (sarcoma) or glial origin, a solid tumour including refractory prostate and ovarian cancer, melanoma and Kaposi's sarcoma, small cell lung cancer, chronic myelogenous leukaemia (CML) or T-cell lymphomas.

A yet further aspect is the use of a compound identified or identifiable by the screening method of the invention in the manufacture of a medicament for the treatment of inflammation, disorders of bone growth or homeostasis (such as osteoporosis), arthritis or atherosclerosis wherein the compound is not a pyridinylimidazole inhibitor (for example SB 203580 or as set out in WO 95/02591).

The compounds may be administered in any suitable way, usually parenterally, for example intravenously, intraperitoneally or intravesically, in standard sterile, non-pyrogenic formulations of diluents and carriers. The compounds of the invention may also be administered topically, which may be of particular benefit for treatment of surface wounds. The compounds of the invention may also be administered in a localised manner, for example by injection.

It will be appreciated that a further aspect of the invention is the compounds identified or identifiable by the screening method of the invention for use in medicine.

A further aspect of the invention is a method of treatment of a patient in need of inhibition of a protein kinase that has a threonine or less bulky residue at the position equivalent to Thr 106 of SAPK2a/p38, wherein the protein kinase is not SAPK2a/p38 or SAPK2b/p38β2, comprising administering an effective amount of a pyridinylimidazole inhibitor or related inhibitor or a compound identified or identifiable by the screening method of the invention.

Thus, a further aspect of the invention is a method of treatment of a patient in need of inhibition of a type-I or type-Il TGFβ receptor, comprising administering an effective amount of SB 203580. Such a patient may be a patient with a disease or condition in which TGFβ has been implicated, for example as set out in the methods of treatment described below.

A still further aspect is a method of treatment of a patient in need of reducing extracellular matrix deposition, encouraging tissue repair and/or regeneration, tissue remodelling or healing of a wound, injury or surgery, or reducing scar tissue formation arising from injury to the brain, comprising administering an effective amount of a pyridinylimidazole inhibitor or related inhibitor or a compound identified or identifiable by the screening method of the invention.

A still further aspect is a method of treatment of a patient with or at risk of end-stage organ failure, pathologic extracellular matrix accumulation, disease states associated with immunosuppression (such as different forms of malignancy, chronic degenerative diseases, and AIDS), diabetic nephropathy, tumour growth, kidney damage or renal fibrosis comprising administering an effective amount of a pyridinylimidazole inhibitor or related inhibitor or a compound identified or identifiable by the screening method of the invention.

It will be appreciated that forms of chronic degenerative diseases in which excessive IL-1, IL-8 or TNF production is implicated are not disease states associated with immunosuppression, and are therefore excluded from the definition. Thus forms of multiple sclerosis or muscle degeneration in which excessive IL-1, IL-8 or TNF production is implicated are excluded. Similarly, forms of AIDS in which excessive IL-1, IL-8 or TNF production is implicated are excluded.

A still further aspect is a method of treatment of a patient with a cancer of epithelial origin, for example breast, lung, ovary, colon or prostate, a cancer of mesenchymal (sarcoma) or glial origin, a solid tumour including refractory prostate and ovarian cancer, melanoma and Kaposi's sarcoma, small cell lung cancer, chronic myelogenous leukaemia (CML) or a T-cell lymphoma comprising administering an effective amount of a pyridinylimidazole inhibitor or related inhibitor or a compound identified or identifiable by the screening method of the invention.

The invention is now described in more detail by reference to the following, non-limiting, Figures and Examples:

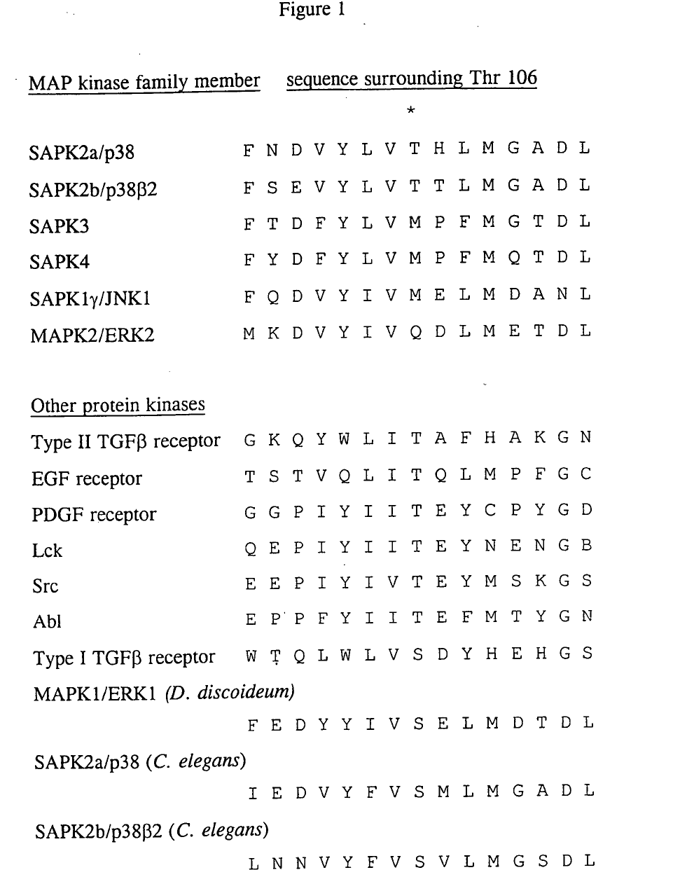

FIG. 1: Amino acid sequences surrounding the threonine residue in SAPK2a/p38 and SAPK2b/38β2 that confers sensitivity to SB 203580.

(A) Human MAP kinase family members; (B) Protein kinases with Thr or Ser in the position equivalent to Thr 106 of human SAPK2a/p38. The residues in the position equivalent to Thr 106 in human SAPK2a/p38 are marked by an asterisk.

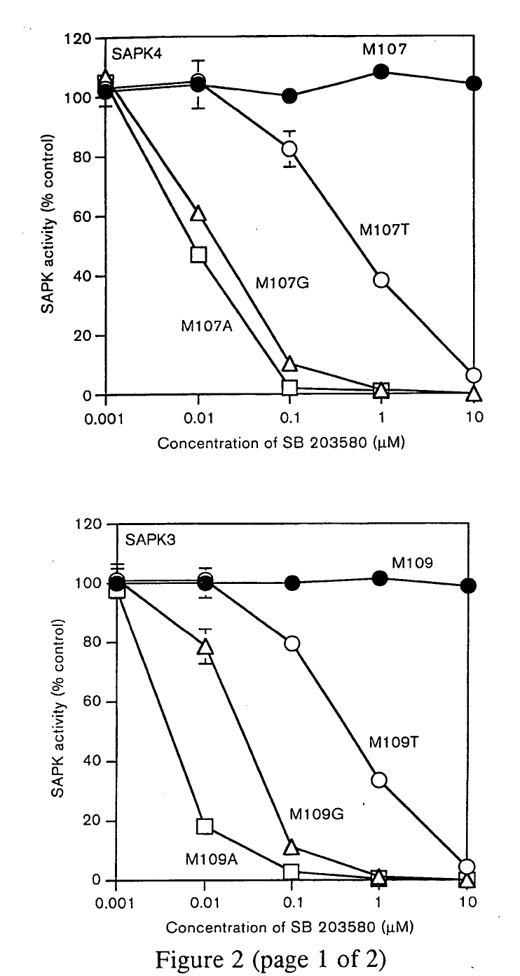

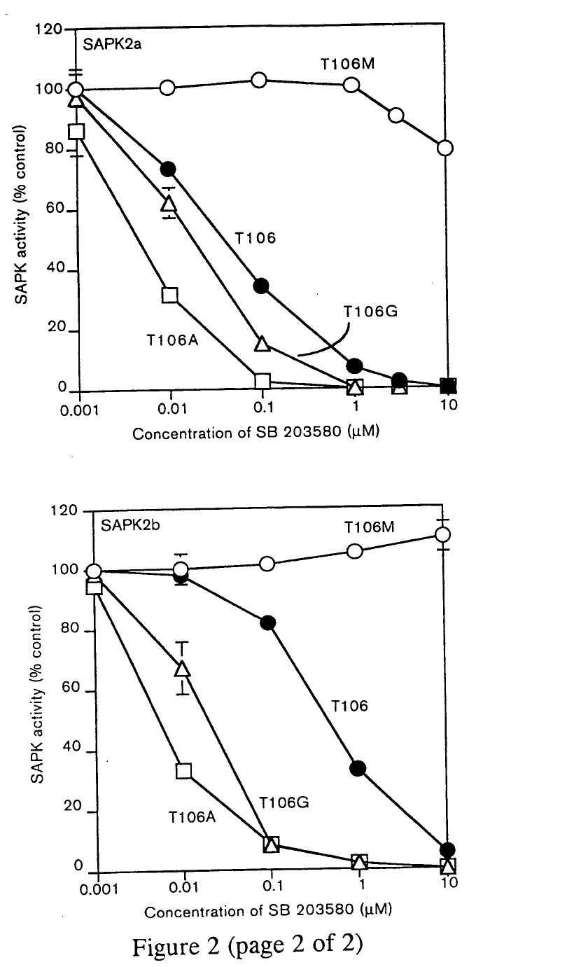

FIG. 2: Inhibition of wild-type and mutant SAPK2a/p38, SAPK2b/p38β2, SAPK3 and SAPK4 by SB 203580.

The effect of the drug on the wild-type enzymes is shown by the closed circles and its effect on mutant enzymes by open symbols. Wild-type SAPK3 and SAPK4 are resistant to SB 203580, because they have Met at positions 109 and 107, respectively. Wild-type SAPK2b/p38β2 has Thr at position 106 and is inhibited by SB 203580, with an IC₅₀ value of 1 μM. Met 106 SAPK2b/p38β2 is resistant to SB 203580. Wild-type SAPK2a/p38 has Thr at position 106 and is inhibited by SB 203580, with an IC₅₀ value of 0.08 M. Met 106 SAPK2a/p38 is still inhibited by SB 203580, but the IC₅₀ value is now 100 μM.

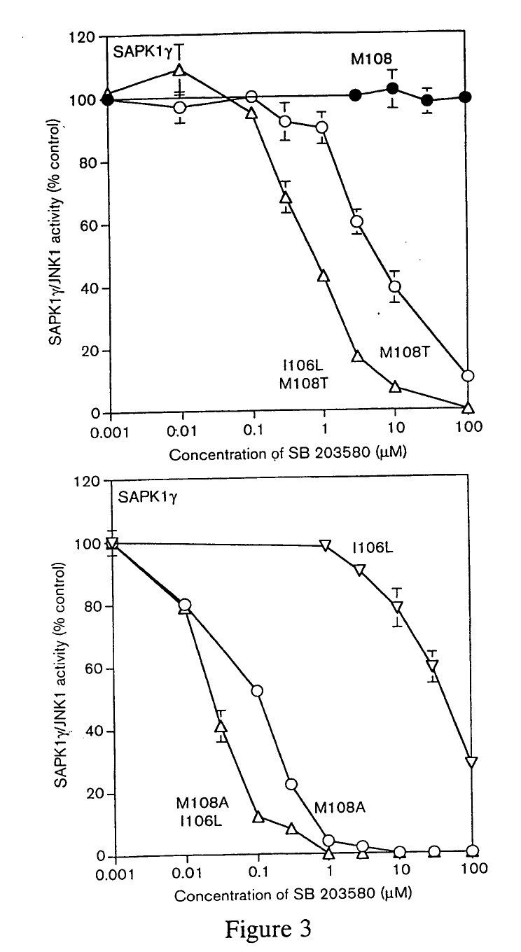

FIG. 3. Inhibition of wild-type and mutant SAPK1y/JNK1 by SB 203580.

The effect of the drug on the wild-type enzyme is shown by the closed circles and its effect on the mutant enzymes by open symbols. Wild-type SAPK1γ/JNK1 is resistant to SB 203580, because it has Met at position 108 and Ile at position 106.

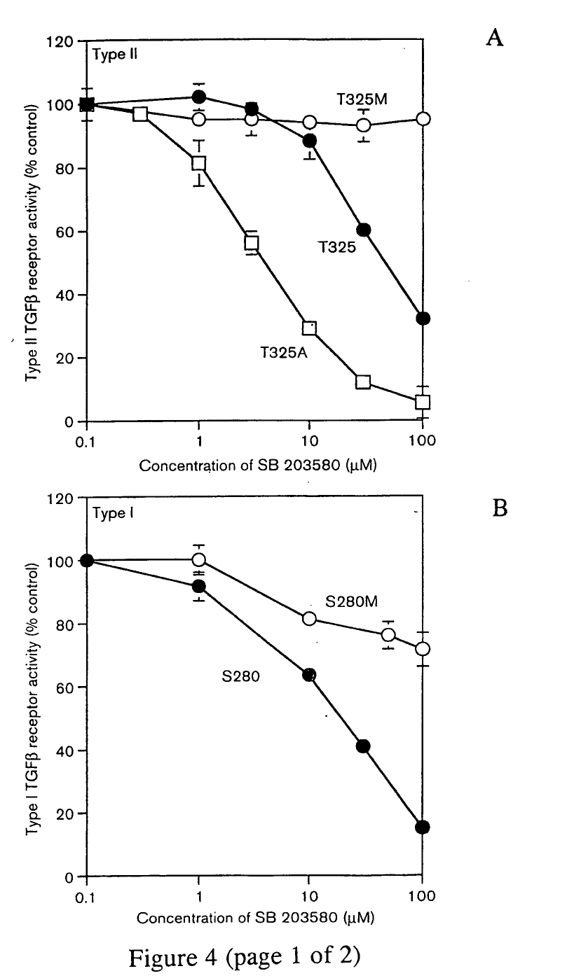

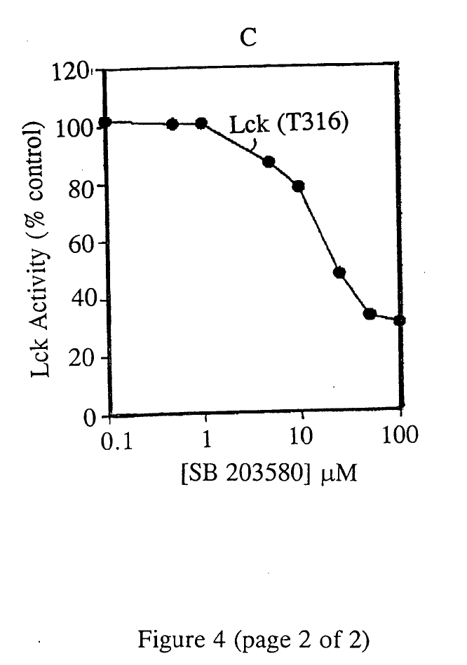

FIG. 4. Inhibition of type-I TGFβ receptor, type-II TGFβ receptor and tyrosine protein kinase Lck by SB 203580.

(A) Inhibition of the type-II TGFβ receptor. The effect of SB 203580 on the native type-II TGFβ receptor is shown by closed circles and its effect on the mutant enzymes by open symbols. The type-II TGFβ receptor has a Thr at position 325 and is inhibited by SB 203580, with an IC₅₀ value of 40 μM. The Thr 325Ala mutant is inhibited with an IC₅₀ of 4 μM, while the Thr325Met mutant is resistant to SB 203580.

(B) Inhibition of the type-I TGFβ receptor. The effect of SB 203580 on the native type-1 TGFβ receptor is shown by closed circles and its effect on the Ser280Met mutant by open circles. This type-I TGFβ receptor has Ser at position 280 and is inhibited by SB 203580 with an IC₅₀ of 20 μM. Replacement of this Ser with Met increases the IC₅₀ value to >100 μM.

(C) Inhibition of Lck. The effect of the drug is shown by closed circles. Lck has a Thr at position 316 and is inhibited by SD 203580, with an IC₅₀ value of 20 μM.

FIG. 5: generic structure of pyridinylimidazole inhibitors

FIG. 6: structures of particular pyridinylimidazole inhibitors

FIG. 7: alignment of partial sequences of TGFβ family receptors.

FIG. 8: protein tyrosine kinase sequences flanking the residue equivalent to Thr 106 in SAPK2a/p38. The sequences are from records held in the Prints Database 17.0 and the information shown is sufficient to identify the records from which the sequences are derived.

### EXAMPLE 1

### Conversion of SB 203580-Insensitive MAP Kinase Family Members to Drug-Sensitive Forms by a Single Amino Acid Substitution Reveals a New Paradigm for the Development of Specific Protein Kinase Inhibitors

Specific inhibitors of protein kinases have great therapeutic potential, but the molecular basis underlying their specificity is only poorly understood. We have investigated the drug SB 203580, which belongs to a class of pyridinyl imidazoles that inhibits the stress-activated protein (SAP) kinases SAK2a/p38 and SAPK2b/p38β2, but not other mitogen-activated protein (MAP) kinase family members. Like specific inhibitors of other protein kinases, SB 203580 binds in the ATP-binding pocket of SAPK2a/p38. The SAP kinases SAPK1/JNK, SAPK3 and SAPK4 are not inhibited by SB 203580, because they have methionine in the position equivalent to threonine 106 in the ATP-binding region of SAPK2a/p38 and SAPK2b/p38β2. Using site-directed mutagenesis of five SAP kinases and the type-I and type-II TGFβ receptors, we establish that for a protein kinase to be inhibited by SB 203580, the side-chain of this residue must be no larger than that of threonine. Sensitivity to inhibition by SB 203580 is greatly enhanced when this side-chain is even smaller, as in serine, alanine and glycine. Thus, the type-I TGFβ receptor, which has serine at the position equivalent to threonine 106 of SAPK2a/p38 and SAPK2b/p38β2, is inhibited by SB 203580.

These findings explain how drugs that target the ATP-binding site can inhibit protein kinases specifically and show that the presence of Thr or a smaller amino acid is diagnostic of whether a protein kinase is sensitive to this class of drug.

Protein kinases form one of the largest families of proteins encoded by the human genome, and play pivotal roles in almost all aspects of cell regulation. Abnormal protein phosphorylation is the cause or consequence of many diseases and, for this reason, protein kinases have become attractive targets for drug therapy. Several relatively specific inhibitors of these enzymes have been developed that have therapeutic potential for the treatment of cancer, diabetes, hypertension and inflammation [1]. A class of pyridinyl imidazoles suppresses the synthesis (and some of the actions) of pro-inflammatory cytokines, and shows promise for the treatment of rheumatoid arthritis and other chronic inflammatory conditions [2]. These compounds are remarkably specific inhibitors of stress-activated protein (SAP) kinase 2a (SAPK2a, also termed p38, RK and CSBP2) and SAP kinase 2b (SAPK2b. also termed p38β2) [3] and have been used to identify physiological substrates for these enzymes, such as the protein kinases MAPKAP-kinases 2/3 and Mnk1/2, and several transcription factors (reviewed in [4]).

SAPK2a/p38 and SAPK2b/p38β2, which show 74% sequence identity, are members of the mitogen-activated protein (MAP) kinase family.

However, other members of this family whose amino acid sequences are 40-60% identical to SAPK2a/p38 and SAPK2b/p38β2 are not inhibited by the pyridinyl imidazoles SB 203580 or SB 202190 [3, 5-7]. These enzymes include SAPK1 (or JNK), which consists of a number of closely related isoforms that phosphorylate the transactivation domain of c-Jun [6] and the more recently identified SAPK3 (also called ERK6 and p38γ) [7] and SAPK4 (also called p38δ) [3, 5] whose physiological substrates are unknown.

SB 203580 binds competitively with ATP, and the three-dimensional structure of SAPK2a/p38 in a complex with a closely related pyridinyl imidazole has established that these drugs are inserted into the ATP-binding pocket of SAPK2a/p38 [8]. These analyses also revealed that the 4fluorophenyl ring of the drug does not make contact with residues in the ATP-binding pocket that interact directly with ATP. One residue near the 4-fluorophenyl ring is Thr 106 and mutation of this residue to Met makes SAPK2a/p38 insensitive to the drug [8]. Thr106 is conserved in SAPK2b/p38β2, but replaced by Met in SAPK1/JNK, SAPK3 and SAPK4 (FIG. 1). We therefore examined the role played by this Thr/Met residue in conferring sensitivity/resistance to these and other protein kinases. SAPK1/JNK, SAPK3 and SAPK4 became sensitive to SB 203580 following mutation of the Met at this position to Thr or a smaller amino acid. The presence of Ser or Thr at this position was also found to be diagnostic of whether more distantly related protein kinases are sensitive to inhibition by SB 203580.

SAPK3 and SAPK4 both became sensitive to SB 203580, when Met in the equivalent position of Thr 106 in SAPK2a/p38 was changed to Thr (FIG. 2) [9]. Further mutagenesis to a variety of other residues revealed that a side chain smaller than that of threonine, as in Ser, Ala and Gly, made SAPK3 more sensitive to the drug, while inhibition was extremely poor when a large hydrophobic or charged residue was present at this position, such as Gln (Table 1), which is found at this position in the MAP kinase family members ERK1 and ERK2. The Met109Ala and Met109Gly mutants of SAPK3 had no significant effect on the K_{CAT}/K_{M} values, when compared with the wild-type enzyme (data not shown).

SAPK4, SAPK2a/p38 and SAPK2b/p38β2 also became most sensitive to SB 203580, when Gly or Ala was present at this position (IC₅₀=15-30 nM) (FIG. 2). Human wild-type SAPK2a/p38 was inhibited about 10-fold more potently than human wild-type SAPK2b/p38β2 or the Thr mutants of SAPK3 and SAPK4 (FIG. 2). The sensitivities of all these enzymes to SB 203580 became similar, however, after mutation of Thr 106 to Gly or Ala (FIG. 2). The Ala mutants were inhibited more strongly than the Gly mutants, suggesting that the size of the alanine side-chain is optimal for inhibition by SB 203580.

TABLE 1
Effect of mutating Met 109 on the
sensitivity of SAPK3 to SB 203580

| Residue at position 109 | IC₅₀ value (μM) |
|---|---|
| Methionine | >100 |
| Phenylalanine | >100 |
| Lysine | 55 |
| Glutamine | 50 |
| Leucine | 45 |
| Glutamate | 45 |
| Threonine | 0.3 |
| Serine | 0.05 |
| Glycine | 0.03 |
| Alanine | 0.01 |

Site-directed mutagenesis was used to change residue 109 in SAPK3 from methionine to one of nine other amino acids.

SAPK2a/p38 and SAPK2b/p38β2 are less similar to the isoforms of SAPK1/JNK (40% identity) than to SAPK3 or SAPK4 (60% identity) [2, 5-7]. Nevertheless, SAPK1-γ/JNK1 became sensitive to inhibition by SB 203580 when Met 108 was mutated to Thr (FIG. 3), although the IC₅₀ value (10 μM) was 30- to 50-fold higher than for SAPK2b/p38β2 or the Thr mutants of SAPK3 or SAPK4, and 500-fold higher than for wild-type SAPK2a/p38 (FIG. 2). However, after mutation to Ala, SAPK1γ/JNK1 was inhibited by SB 203580 at submicromolar concentrations (FIG. 3). SAPK1γ/JNK1 differs from other SAP kinases by the presence of Ile at position 106 instead of Leu. When residue 106 was changed to Leu in the wild-type enzyme, SAPK1γ/JNK1 was inhibited to some extent by SB 203580, with an IC₅₀ value of 50 μM (FIG. 3). When Ile 106 was changed to Leu in the Thr 108 and Ala 108 mutants, SAPK1γ/JNK1 was strongly inhibited by SB 203580, with an IC₅₀ value of 30 nM for Leu 106 Ala 108 SAPK1γ/JNK1 (FIG. 3). These results show that residue 108 is the major determinant for inhibition of SAPK1γ/JNK1 by pyridinyl imidazoles and that residue 106 plays a minor role.

Thr 106 of SAPK2a/p38 is located in subdomain IV of the kinase catalytic domain (FIG. 1). Examination of the sequences of other protein kinases revealed that a bulky hydrophobic residue is almost always found at this position. Nevertheless, a small number of protein kinases do have Thr at this position, such as the type-II TGFβ receptor (a serine/threonine protein kinase), members of the Src family of protein tyrosine kinases and some receptor protein tyrosine kinases, such as the EGF and PDGF receptors (FIG. 1). In order to see whether the presence of Thr at this position is diagnostic of sensitivity to SB 203580, we examined whether the type-II TGFβ receptor and Lck (a Src family member) were inhibited by this drug [11]. These experiments revealed that both kinases were inhibited by SB 203580, with IC₅₀ values of 20 μM (Lck) and 40 μM (type-II TGFβ receptor) (FIG. 4). When Thr residue 325 in the type-II TGFβ receptor was changed to Met, the enzyme became insensitive to the drug (FIG. 4). In addition, SK&F 105809, a closely related pyridinyl imidazole that does not inhibit SAPK2a/p38 (3), did not inhibit the type-II TGFβ receptor. The type-II TGFβ receptor was inhibited with the same IC₅₀ value whether it was assayed by autophosphorylation (FIG. 4) or using histone H2B as a substrate (data not shown). These results establish that the sensitivity of the type-II TGFβ receptor to SB 203580 (FIG. 4) is conferred by Thr residue 325 in subdomain IV.

Very few protein kinases have Ser at the position of Thr 106 of SAPK2a/p38 (for instance a mammalian type-I TGFβ receptor isoform, SAPK2a/p38 and SAPK2b/p38P from Caenorhabditis elegans and MAP kinase from Dictyostelium discoideum [12]) (FIG. 1). In order to see if the presence of Ser at this position confers sensitivity to SB 203580, we examined whether the type-I TGFβ receptor was inhibited by this compound [13]. As shown in FIG. 4, the type-I TGFβ receptor was sensitive to inhibition by SB 203560, with an IC₅₀ value of 20 μM. By contrast, when Ser280 in the type-I TGFβ receptor was changed to Met, the enzyme became insensitive to the drug. These finding are consistent with the mutagenesis study on SAPK3 which showed that the Ser 109 mutant was more sensitive to inhibition by SB 203580 than the Thr 109 mutant (Table 1).

The present findings demonstrate that inhibition of SAPK2a/p38 and SAPK2b/p38β2 by the pyridinyl imidazole SB 230580 depends on the size of the amino acid residue at position 106 in the ATP-binding region. Mutation of this residue from Thr to Met made SAPK2b/p38β2 insensitive to SB 203580. It also rendered SAPK2a/p38 almost completely resistant to SB 203580, in agreement with a previous study [8]. The failure of the Thr to Met mutation to totally abolish inhibition of SAPK2a/p38 by SB 203580 (IC₅₀ of 100 μM) suggests that another residue(s) may contribute to the extremely potent inhibition of wild-type SAPK2a/p38 by SB 203580 (FIG. 2). For example, SAPK2b/p3802, which is 10-fold less sensitive to SB 203580 than SAPK2a/p38 (FIG. 2), differs from SAPK2a/p38 at residues 100, 101 and 107 (FIG. 2). However, the sequences of human and X laevis SAPK2a/p38 are identical in this region, despite the latter being at least 5-fold less sensitive to SB 203580 [3].

Conversely, mutation of the equivalent residue in SAPK3 and SAPK4 from Met to Thr rendered these enzymes sensitive to inhibition by SB 203580. Further mutagenesis revealed that SAPK3/p38γ became mores sensitive to the drug, when Met was replaced by even smaller residues, such as Ser, Ala or Gly. SAPK2a/p38, SAPK2b/p38β2 and SAPK4/p38β also became most sensitive to SB 203580 when Gly or Ala was present at this position. These findings rule out the possibility that the inhibition of SAPK2a/p38 and SAPK2b/p38β2 results from the formation of a hydrogen bond between the hydroxyl group of Thr 106 and the 4-fluorophenyl moiety of SB 203580. SAPK1/JNK, another MAP kinase family member, is only 40% identical to SAPK2a/p38 and SAPK2b/p38β2. Nevertheless, SAPK1/JNK could also be converted to a SB 203580-sensitive form by mutation of Met 108 to a small amino acid. However, in order to optimise sensitivity to the drug, it was also necessary to change Ile 106 to the Leu residue present at the equivalent position in other MAP kinase family members. The latter may explain the reported inhibition of JNK2 βl and JNK2βby high concentrations of a pyridinyl imidazole compound [10], since these isoforms of SAPKI/JNK have a Leu at position 106 and a Met at position 108 [6].

Most known mammalian protein kinases have a large, hydrophobic, residue at the position equivalent to Thr 106 of SAPK2a/p38. Few protein kinases have Thr at this position and only two (the type-I TGFβ receptor and a type-I activin receptor) have Ser. Two protein kinases with Thr (the type-TGFβ receptor and the tyrosine protein Innase Lck) were found to be sensitive to inhibition by SB 203580, although the IC₅₀ values were 400-800 times higher than the IC₅₀ value for SAPK2a/p38, indicating that residues other than the one equivalent to Thr106 of SAPk2a/p38 may also play a role. Nevertheless, sensitivity of the type-II TGFβ receptor to SB 203580 was enhanced by mutagenesis of Thr325 to Ala, whereas it was abolished when this residue was changed to Met. The type-I TGFβ receptor was inhibited more potently by SB 203580 than the type-II TGFβ receptor, consistent with the presence of the smaller Ser residue at this position.

When studying phenotypic effects of SB 203580 in cells, we have emphasised previously the importance of establishing that they occur at a concentration similar to that which prevents the activation of SAPK2a/p38 in the same cell type [4]. The realisation that other protein kinases containing Ser or Thr are inhibited by SB 203580 at concentrations above 10 μM makes this experiment mandatory. It is also important to see whether all the reported effects of SB 203580 in mammalian cells are abolished by transfection with a drug-insensitive form of SAPK2a/p38 or by the use of transgenic mice expressing an SB 203580-insensitive form of this protein kinase.

No known protein kinases have an Ala or Gly at the position of Thr 106 of SAPK2a/p38. If such enzymes exist, they can be expected to be potently inhibited by SB 203580. Recent work on non-receptor tyrosine kinases of the Src family has shown that the size of the residue equivalent to Thr 106 in SAPK2a/p38 is also primarily responsible for determining the ability of protein kinases to accept N⁶ substituted ATP analogues [14], further emphasizing the crucial role that this residue plays in determining the size of the ATP-binding pocket of protein kinases.

Nearly all eukaryotic kinases belong to the same protein superfamily and their ATP binding sites are similar. It therefore seemed unlikely that specific inhibitors of particular protein kinases could be developed by targeting this region of these enzymes. Recent work describing a new class of receptor protein tyrosine kinase inhibitor suggests that this fear may be unfounded [15]. In addition, the present work indicates that remarkable specificity can be achieved if drugs that interact with the ATP-binding pocket carry an additional moiety that makes contact with residues outwith this region.

Our finding that sensitivity to SB 203580 can be gained by a single amino acid substitution means that it should be possible to exploit the same drug to identify the physiological roles of all MAP kinase family members and other protein kinases. For example, it will be interesting to see whether all the reported effects of SB 203580 in mammalian cells are abolished by transfection with a drug-insensitive form of SAPK2a/p38 or by the use of transgenic mice expressing an SB 203580-insensitive form of this protein kinase. The further replacement of wild-type SAPK1/JNK, SAPK3 or SAPK4 by drug-sensitive forms of these enzymes in transgenic mice expressing a drug-resistant form of SAPK2a/p38 may also be useful in addressing the physiological roles of these other MAP kinase family members.

Finally, our results show that it is possible to predict whether a protein kinase will be sensitive to inhibition by this class of pyridinyl imidazoles, simply by inspection of the amino acid at the position equivalent to Thr 106 of SAPK2a/p38. It may therefore be possible to identify ‘lead’ inhibitors for a number of protein kinases, without any high throughput screening.

Significance

Protein kinases are involved in specific ways in most physiological processes and abnormal phosphorylation of proteins is an essential feature of many disease states. It is widely believed that specific protein kinase inhibitors will be some of the drugs of the future, permitting improved treatment of a large number of serious diseases. It is therefore critical to understand the mechanisms of action of the few existing protein kinase inhibitors with a high degree of specificity. The pyridinyl imidazole SB 203580 is such an inhibitor. It inhibits stress-activated protein (SAP) kinase-2a (SAPK2a, also called p38) and SAP kinase-2b (SAPK2b, also called p38β2), but not other members of the mitogen-activated protein (MAP) kinase family. SB 203580 has been used to identify some of the physiological substrates and cellular functions of SAPK2a/p38 and SPAK2b/p38β2. Here we show that for a protein kinase to be sensitive to SB 203580, the amino acid at the position equivalent to residue 106 of SAPK2a/p38 must be no larger than threonine, the sensitivity being greatly enhanced when this residue is serine, alanine or glycine. The high degree of specificity of SB 203580 results from the presence of a residue larger than threonine at this position in nearly all known protein kinases. These findings explain how drugs that target the ATP-binding site can inhibit protein kinases specifically and show that it is possible to predict whether a protein kinase will be sensitive to inhibition by this class of pyridinyl imidazoles, simply by inspection of the amino acid at the position equivalent to threonine 106 of SAPK2a/p38 in subdomain IV of the kinase catalytic domain. Our data will facilitate the rational design of specific protein kinase inhibitors.

### REFERENCES AND NOTES

1. N. M. Low and N. B. Lydon (1996) In Emerging Drugs: The Prospect of Improved Medicines pp. 241-260 (Ashley Publications Ltd.); H. Ishi et al., Science 272, 728 (1996): M. Uehata et al., Nature 369, 1390 (1997); A. M. Badger et al., J. Pharmacol. Exp. Ther. 279, 1458 (1996).
2. J. C. Lee et al., Nature 372, 739 (1994).
3. A. Cuenda et al., FEBS Lett. 364, 229 (1995); M. Goedert, A. Cuenda, M. Craxton, R. Jakes, P. Cohen, EMBO J. 16, 3563 (1997).
4. P. Cohen, Trends Cell Biol. 7, 353 (1997).
5. S. Kumar et al., Biochem. Biophys. Res. Commun. 235, 533 (1997); B. X. Wang et al. J. Biol. Chem. 272, 23668 (1997); Y. Jiang et al., J. Biol, Chem. 272, 30122 (1997).
6. B. Dérljard et al., Cell 76, 1025 (1994); S. Gupta et al. EMBO J. 15, 2760 (1995).
7. S. Mertens, M. Craxton, M. Goedert FEBS Lett. 383, 273 (1996); C. Lochner, M. A. Zahalka, J.-F. Glot, N. P. H. Moller, A. Ulrich, Proc. Natl. Acad. Scl, USA 93, 4355 (1996); Z. U, Y. Jiang, R. J. Ulevitch, J. Han, Biochem. Biophys. Res. Commun. 213, 715 (1996); A. Cuenda, P. Cohen, V. Buée-Scherrer, M. Goedert, EMBO J. 16, 295 (1997).
8. P. R. Young et al., J. Biol. Chem. 272, 12116 (1997). L. Tong et al., Nature Struct. Biol. 4, 311 (1997); K. P. Wilson et al., Chem. Biol. 4, 423 (1997).

9. Full-length cDNA clones encoding human SAPK1γ/JNK1. human SAPK2a/p38 (CSBP2 isoform), human SAPK2b/p38β2, rat SAPK3 and human SAPK4 were subcloned into M13. Site-directed mutagenesis was used to change Ile 106 in SAPK1y/JNK1 to Leu and/or Met 108 to Ala or Thr; Thr 106 in SAPK2a/p38 was changed to Ala, Gly or Met; Thr 106 in SAPK2b/p38β2 was changed to Ala, Gly or Met; Met 109 in SAPK3 was changed to Ala, Gln, Glu, Gly, Leu, Lys, Phe, Ser or Thr; Met 107 in SAPK4 was changed to Ala, Gly or Thr. All mutations were verified by DNA sequencing. Following primer-extension, the mutated cDNAs were subcloned into bacterial expression vectors pRSETB (Invitrogen) or pGEX4T-1 (Pharmacia) and expressed as His-tagged protein (SAPK1γ/JNK1) or as GST-fusion proteins (SAPK2a/p38, SAPK2b/p38β2, SAPK3 and SAPK4) in E. coli BL21(DE3) calls. Wild-type SAPK1γ/JNK1 and its mutants were activated using bacterially expressed SAPK kinase-1 (SKK1, also called MKK4 or SEK1) which had been activated by MEK kinase-1 and assayed at 30° C. in the presence and absence of the indicated concentrations of SB 203580 using GST-ATF2(19-96) as substrate. The concentration of ATP in the assays was 0.1 mM. Wild-type SAPK2a/p38, SAPK2b/p38β2, SAPK3 and SAPK4 and their respective mutants were purified on glutathione-Sepharose and activated by a partially active mutant of SAPK kinase-3 (SKK3, also called MKK6), in which Ser 274 and Thr 278 had been mutated to Asp. This constitutively active SKK3/MKK6 mutant was expressed as a maltose-binding protein fusion protein in E. coli and purified on an amylose resin. Each SAP kinase was assayed at 30° C. in the presence and absence of the indicated concentrations of SB 203580 using myelin basic protein as substrate, as described for p42 MAP kinase [D. R. Alessi et al., Meth. Enzymol, 255, 279 (1995)]. The concentration of ATP in the assays was 0.1 mM. Mutants of SAPK1γ/JNK1, SAPK2a/p38, SAPK2b/p38β, SAPK3 and SAPK4 were activated at the same rates and to the same specific activities as the wild-type enzymes. SB 203580 was purchased from Calbiochem.
10. A. J. Whitmarsh, S.-H. Yang, M, S.-S. Su, A. D. Sharrocks, R. J. Davis, Mol. Cell. Biol. 17, 2360 (1997).
11. Human Lck [J. M. Trevllyan et al., Biochem. Biophys. Acta 888, 286 (1986)] was expressed in Sf9 cells from 2 recombinant baculoviral vector and assayed using the peptide KVEKIGPGTYGVVYK, in the presence and absence of the indicated concentrations of SB 203580. Site-directed mutagenesis was used to change Thr 325 in the type-II TGFβ receptor (H. Y. Lin, X. F. Wang. E. Ng-Eaton, R A. Weinberg, H. F. Lodish, Cell 68, 775 (1992)] to Met. Mutagenesis was carried out as described [R. W. MacKintosh et al., FFBS Lett 371, 236 (1995)]. The C-terminal 375 residues of wild-type TGFβ type-II receptor and the corresponding mutants were expressed in E. coli as GST-fusion proteins and purified on glutathione-Sepharose [S. Lawler et al., J. Biol. Chem. 272, 14850 (1997)]. The enzymes were assayed at 30° C. in the presence and absence of the indicated concentrations at SB 203580 or SK&F 105809 by the rate of autophosphorylation or using histone H₂B (0.1 mg/ml) as substrate. The concentration of ATP in the assays was 0.1 mM. The mutants of the type-II TGFβ receptor had the same specific activity as the wild-type enzyme.
12. P. Franzén et al., Cell 75, 681 (1993); Genbank Accession Numbers of the C. elegans sequences: 1326389 for SAPK2a/p38 and 2702444 for SAPK2b/p38β. These sequences were determined by the C. elegans Genome Consortium [R. Wilson et al., Nature 368, 32-38 (1994)]; C. Gaskins, M. Maeda, R. A. Firtel, Mol, Cell. Biol. 14, 6996 (1994).
13. Site directed mutagenesis was used to change Ser280 in the type-I TGFβ receptor (Franzén et al (1993) Cell 75, 681-692) to Met. The C-terminal 356 residues of the wild-type and mutant type-I TGFβ receptors were expressed in E. coli as a GST-fusion protein and purified on glutathione-Sepharose. The enzyme was assayed by the rate of autophosphorylation at 30° C. in the presence or absence of the indicated concentrations of SB 203580. The concentration of ATP was 0.1 mM.
14. Y. Liu, K. Shah, F. Yang, L. Witucki, K. M. Shakat, Chem. Biol., in press.
15. M. Mohammadi et al., Science 276, 955 (1997).
Han et al (1994) “A MAP kinase targeted by endotoxin and hyperosmolarity in mammalian cells” Science 265, 808-811.
Rouse et al (1994) “A novel kinase cascade triggered by stress and heat shock that stimulates MAPKAP kinase-2 and phosphorylation of the small heat shock proteins” Cell 78, 1027-1037.
Stein et al (1997) “p38-2, a novel mitogen-activated protein kinase with distinct properties” J Biol Chem 272, 19509-19517.
Wang et al (1997) “Molecular cloning and characterisation of a novel p38 mitogen-activated protein kinase” J Biol Chem 272, 23668-23674.
Jiang et al (1997) “Characterisation of the structure and function of the fourth member of p38 group mitogen-activated protein kinase, p38δ” J Biol Chem 272, 30122-30128.
Whitmarsh et al (1997) “Role of p38 and JNK mitogen-activated protein kinases in the activation of ternary complex factors” Mol Cell Biol 17, 2360-2371.

### EXAMPLE 2

### Identification of a Protein Kinase for which a Pyridinylimidazole is a Potential Inhibitor

A protein kinase is identified by a protein sequence database (for example, Genbank) search detecting sequence similarity with a known protein kinase or protein kinases. By computer or visual alignment with one or more protein kinase sequences, including that of SAPK2a/p38, the amino acid residue corresponding to Thr 106 in SAPK2a/p38 is identified. The type of this residue is assessed. If it is an amino acid residue that is more bulky than Thr, then the protein kinase is unlikely to be inhibited by a pyridinylimidazole inhibitor with a 4-phenyl ring, for example SB 203580. If it is Thr or a less bulky amino acid, for example Ser, Ala or Gly, then the protein kinase is likely to be inhibited by a pyridinylimidazole inhibitor with a 4-phenyl ring, such as SB 203580.

Pyridinylimidazole inhibitors are then tested for the ability to bind to and/or inhibit the protein kinase. This is carried out by known means of detecting interactions between a protein and a small molecule or by measuring the enzymatic activity of the protein kinase (which is preferably tested when it is in an activated form) by known means.

If the substrate of the protein kinase is not known, its activity against a selection of known substrates for protein kinases may be measured in order to determine a suitable test substrate.

### EXAMPLE 3

### Optimisation of Inhibition of a Protein Kinase by a Pyridinylimidazole Inhibitor

Once a protein kinase has been found to be inhibited by a pyridinylimidazole inhibitor, the inhibitor compound may be modified in order to achieve one or more of the following:
a) increase the potency of the inhibition of the protein kinase under consideration ie reduce the IC₅₀ of inhibition of the protein kinase under the assay conditions,
b) decrease the potency of inhibition of other protein kinase(s),
c) increase the relative potency of inhibition of the protein kinase under consideration compared to inhibition of other protein kinase(s),
d) improve the pharmacological profile of the compound (bioavailability/stability/toxicity).

The properties of the compound are measured in in vitro assays (isolated enzyme or whole cell based) or in vivo assays, which include animal models of disease states.

1. A method of inhibiting a protein kinase that has a threonine or less bulky residue at the position equivalent to Thr 106 in SAPK2a/p38 wherein the protein kinase is exposed to a pyridinylimidazole inhibitor or related inhibitor, provided that the protein kinase is not mammalian SAPK2a/p38 or SAPK2b/p38β2.

2. Use of a pyridinylimidazole inhibitor or related inhibitor in a method of inhibiting a protein kinase that has a threonine or less bulky residue at the position equivalent to Thr 106 in SAPK2a/p38 wherein the protein kinase is exposed to a pyridinylimidazole inhibitor or related inhibitor, provided that the protein kinase is not mammalian SAPK2a/p38 or SAPK2b/p38β2.

3. The use or method of claim 1 or 2 wherein the pyridinylimidazole inhibitor is SB 203580.

4. A screening method for identifying a drug-like compound or lead compound for the development of a drug-like compound in which (1) a pyridinylimidazole or related compound is exposed to a protein kinase that has a threonine or less bulky amino acid at the position equivalent to Thr 106 in SAPK2a/p38 and is not mammalian SAPK2a/p38 or SAPK2b/p38β2 and (2) the binding of the compound to the protein kinase is measured or the change in the activity of the protein kinase is measured.

5. The use or method of any one of claims 1 to 4 performed in vitro.

6. A method of determining that a protein kinase is sensitive to a pyridinylimidazole inhibitor, comprising comparing the amino acid sequence or three dimensional structure of the protein kinase with that of SAPK2a/p38 and determining that the protein kinase has a threonine or less bulky residue at the position equivalent to Thr 106 in SAPK2a/p38.

7. The use or method of any one of claims 1 to 6 wherein the protein kinase is a naturally occuring kinase or variant thereof wherein the amino acid at the position equivalent to Thr 106 in SAPK2a/p38 is naturally a threonine or less bulky residue.

8. The use or method of any one of claims 1 to 7 wherein the amino acid at the position equivalent to Thr 106 in SAPK2a/p38 is threonine, serine, alanine or glycine.

9. The use or method of claim 8 wherein the amino acid at the position equivalent to Thr 106 in SAPK2a/p38 is serine.

10. The use or method of any one of claims 1 to 8 wherein the protein kinase is a type-I activin or bone morphogenetic protein (BMP) receptor, type-II TGFβ or activin receptor, Src family member, epidermal growth factor (EGF) receptor or platelet derived growth factor (PDGF) receptor.

11. The use or method of any one of claims 1 to 9 wherein the protein kinase is a type-I TGFβ receptor or type-I activin receptor.

12. The use or method of claim 11 wherein the protein kinase is a type-I TGFβ receptor.

13. Use of a transgenic animal or a transfected cell in a method of determining a physiological role of a protein kinase, wherein the cell or animal comprises a protein kinase that has been mutated at the position equivalent to Thr 106 in SAPK2a/p38.

14. Use of a transgenic animal, or a transfected cell, comprising a protein kinase that has been mutated at the position equivalent to Thr 106 in SAPK2a/p38, in a screening method for identifying a substrate of said protein kinase.

15. A transgenic animal comprising a protein kinase that has been mutated at the position equivalent to Thr 106 in SAPK2a/p38.

16. A compound identifiable by the screening method of any one of claims 4 to 12.

17. The compound of claim 16 for use in medicine.

18. Use of a pyridinylimidazole inhibitor or related inhibitor or a compound according to claim 16 in the manufacture of a medicament for the treatment of a patient in need of inhibition of a protein kinase that has a threonine or less bulky residue at the position equivalent to Thr 106 of SAPK2a/p38, wherein the protein kinase is not SAPK2a/p38 or SAPK2b/p38β2.

19. Use according to claim 18 wherein the protein kinase has a serine residue at the position equivalent to Thr 106 of SAPK2a/p38.

20. Use according to claim 18 wherein the protein kinase is a type-I or type-Il TGFβ receptor.

21. Use of a pyridinylimidazole inhibitor or related inhibitor or a compound according to claim 16 in the manufacture of a medicament for the treatment of a patient in need of reducing extracellular matrix deposition, encouraging tissue repair and/or regeneration, tissue remodelling or healing of a wound, injury or surgery, or reducing scar tissue formation arising from injury to the brain.

22. Use of a pyridinylimidazole inhibitor or related inhibitor or a compound according to claim 16 in the manufacture of a medicament for the treatment of a patient with or at risk of end-stage organ failure, pathologic extracellular matrix accumulation, a fibrotic condition, disease states associated with immunosuppression, diabetic nephropathy, tumour growth, kidney damage or renal fibrosis.

23. Use of a compound according to claim 16 in the manufacture of a medicament for the treatment form of a disorder of bone growth or homeostasis, arthritis or atherosclerosis in which IL-1, IL-6, IL-8 or TNF or a proinflammatory cytokine have not been implicated, but in which TGFβ or a related protein has been implicated in causing or exacerbating the condition.

24. A method of treatment of a patient in need of inhibition of a protein kinase that has a threonine or less bulky residue at the position equivalent to Thr 106 of SAPK2a/p38, wherein the protein kinase is not SAPK2a/p38 or SAPK2b/p38β2, comprising administering an effective amount of a pyridinylimidazole inhibitor or related inhibitor or a compound according to claim 16.

25. A method of treatment of a patient in need of reducing extracellular matrix deposition, encouraging tissue repair and/or regeneration, tissue remodelling or healing of a wound, injury or surgery, or reducing scar tissue formation arising from injury to the brain, comprising administering an effective amount of a pyridinylimidazole inhibitor or related inhibitor or a compound according to claim 16.

26. A method of treatment of a patient with or at risk of end-stage organ failure, pathologic extracellular matrix accumulation, disease states associated with immunosuppression, diabetic nephropathy, tumour growth, kidney damage or renal fibrosis, comprising administering an effective amount of a pyridinylimidazole inhibitor or related inhibitor or a compound according to claim 16.

27. A method of treatment of a patient with inflammation, disorders of bone growth, arthritis or atherosclerosis comprising administering an effective amount of a compound according to claim 16 wherein the compound is not a pyridinylimidazole inhibitor.

28. Use of a pyridinylimidazole inhibitor or related inhibitor or compound according to claim 16 in the manufacture of a medicament for the treatment of a patient with a cancer of epithelial, mesenchymal (sarcoma) or glial origin, a solid tumour, melanoma, small cell lung cancer, chronic myelogenous leukaemia (CML) or a T-cell lymphoma.
